(19) United States
(12) Patent Application Publication
(10) Pub. No.: US 2023/0140879 A1
(43) Pub. Date: May 11, 2023

(54) ELECTROCHEMICAL MEASURING OF REACTIVE OXYGEN SPECIES (ROS) LEVELS IN PERIPHERAL BLOOD TO DETECT RATIO OF LOW-DENSITY NEUTROPHILS (LDNS) TO HIGH-DENSITY NEUTROPHILS (HDNS), SUITABLE TO ALARM PRESENCE OF CANCER IN SUSPICIOUS CASES

(71) Applicants: Mohammad Abdolahad, Tehran (IR); Zohreh Sadat Miripour, Tehran (IR); Mina Amininfar, Tehran (IR)

(72) Inventors: Mohammad Abdolahad, Tehran (IR); Zohreh Sadat Miripour, Tehran (IR); Mina Amininfar, Tehran (IR)

(73) Assignee: Nano Hsegar Sazan Salamat Arya., Tehran (IR)

(21) Appl. No.: 18/091,212

(22) Filed: Dec. 29, 2022

Related U.S. Application Data
(63) Continuation-in-part of application No. PCT/IB2022/051283, filed on Feb. 14, 2022.
(60) Provisional application No. 63/149,282, filed on Feb. 14, 2021.

(51) Int. Cl. G01N 33/487

(52) U.S. Cl. CPC G01N 33/48707

(57) ABSTRACT

A system for real-time detecting cancer by analyzing unprocessed blood. The system includes a sensor, an electrical stimulator-analyzer connected to the sensor, and a processing unit connected to the electrical stimulator-analyzer. The sensor includes three electrodes including a working electrode, a counter electrode, and a reference electrode configured to be put in contact with an unprocessed blood sample. The processing unit is configured to perform a method. The method includes applying a set of voltages between the reference electrode and the working electrode utilizing the stimulator-analyzer, measuring a produced set of currents between the counter electrode and the working electrode utilizing the stimulator-analyzer, measuring a level of a ratio of low-density neutrophils (LDNs) to high-density neutrophils (HDNs) in the unprocessed blood sample by measuring a maximum current of the measured set of currents, and detecting a cancer disease if the measured maximum current is less than a threshold value.

### CROSS-REFERENCE TO RELATED APPLICATION

This application is a continuation-in-part of International Patent Application PCT/IB2022/051283, filed on Feb. 14, 2022, and entitled “ELECTROCHEMICAL MEASURING OF REACTIVE OXYGEN SPECIES (ROS) LEVELS IN PERIPHERAL BLOOD TO DETECT RATIO OF LOW-DENSITY NEUTROPHILS (LDNS) TO HIGH-DENSITY NEUTROPHILS (HDNS), SUITABLE TO ALARM PRESENCE OF CANCER IN SUSPICIOUS CASES”, which takes priority from U.S. Provisional Patent Application Ser. No. 63/149,282, filed on Feb. 14, 2021, and entitled “ELECTROCHEMICAL MEASURING OF REACTIVE OXYGEN SPECIES LEVELS RELEASED BY LOW-DENSITY NEUTROPHILS IN THE BLOOD, AN ALARM OF CANCER IN SUSPICIOUS CASES”, which are both incorporated herein by reference in their entirety.

### TECHNICAL FIELD

The present disclosure generally relates to cancer diagnosis, and particularly, to a system and method for real-time cancer diagnosis by determining a ratio of low-density neutrophils (LDNs) to high-density neutrophils (HDNs) via measuring reactive oxygen species (ROS) levels in unprocessed blood samples.

### BACKGROUND

Neutrophils are the most abundant (50% to 70%) type of white blood cells circulating leukocytes which are derived from hematopoietic stem cells (HSCs) in bone marrow. Neutrophils release enzymes to remodel an extracellular matrix of a tissues through which neutrophils migrate to reach a site of a wound or an infection.

Number of LDN neutrophils in blood and tumor tissues of cancer patients with solid tumors shows an association with disease progression and patient outcome. There are several cell markers for identification of immature neutrophils in human. These markers need further validation to ensure its accuracy. Hence, since neutrophils can show plasticity in response to their environment, certain markers are likely to only be proper in particular models and require efficient validation of them. So, the challenges associated with identifying and isolating populations of neutrophil maturity have limited their study and the current knowledge of their functional properties.

Hence, there is a need for a label-free method and system to determine an accurate correlation between abundancy of neutrophils, specially, LNDs in blood sample of patients with a cancer status of the patients. There is also a need for a highly precise and fast method and system for detecting a cancer status of a suspected patient to be cancerous via measuring a level of LDNs in peripheral blood without any need to process a blood sample and/or use a cell marker.

### SUMMARY

This summary is intended to provide an overview of the subject matter of this patent, and is not intended to identify essential elements or key elements of the subject matter, nor is it intended to be used to determine the scope of the claimed embodiments. The proper scope of this patent may be ascertained from the claims set forth below in view of the detailed description below and the drawings.

In one general aspect, the present disclosure is directed to a method for real-time detecting cancer. The method may include putting an unprocessed blood sample in contact with sensing parts of a set of a working electrode, a counter electrode, and a reference electrode of a sensor, where the unprocessed blood sample may be drawn from a suspected person to have cancer. The method may further include applying a set of voltages in a sweeping range from −0.8 V to +0.8 V between the reference electrode and the working electrode, measuring a produced set of electrical currents between the counter electrode and the working electrode versus the applied set of voltages utilizing one or more processors, measuring a level of ratio of low-density neutrophils (LDNs) to high-density neutrophils (HDNs) in the unprocessed blood sample utilizing one or more processors, and detecting a cancer status of the suspected person based on the measured level of ratio of LDNs to HDNs in the unprocessed blood sample utilizing one or more processors.

In an exemplary embodiment, measuring the level of ratio of LDNs to HDNs in the unprocessed blood sample may include measuring a level of reactive oxygen species (ROS) in the unprocessed blood sample by measuring a maximum electrical current of the measured set of electrical currents and determining the level of ratio of LDNs to HDNs in the unprocessed blood sample based on the measured maximum electrical current of the measured set of electrical currents. In an exemplary embodiment, determining the level of ratio of LDNs to HDNs in the unprocessed blood sample based on the measured maximum electrical current of the measured set of electrical currents may include detecting the level of ratio of LDNs to HDNs in the unprocessed blood sample is more than 1 if the measured maximum electrical current of the measured set of electrical currents is less than a first threshold electrical current value or detecting the level of ratio of LDNs to HDNs in the unprocessed blood sample is less than 1 if the measured maximum electrical current of the measured set of electrical currents is more than a second threshold electrical current value. In an exemplary embodiment, detecting the cancer status of the suspected person may include one of detecting a cancer disease in the suspected person's body if the level of ratio of LDNs to HDNs in the unprocessed blood sample is more than one or detecting no cancer disease in the suspected person's body if the level of ratio of LDNs to HDNs in the unprocessed blood sample is less than one.

In an exemplary embodiment, the method may further include generating the first threshold electrical current value and the second threshold electrical current value. In an exemplary embodiment, generating the first threshold electrical current value and the second threshold electrical current value may include generating a first dataset of a plurality of unprocessed blood samples associated with a plurality of cancer patients, generating a second dataset of a plurality of unprocessed blood samples associated with a plurality of healthy persons, and determining the first threshold electrical current value and the second threshold electrical current value using the first dataset and the second dataset. In an exemplary embodiment, generating the first dataset of the plurality of unprocessed blood samples associated with the plurality of cancer patients may include measuring a first set of electrical current peaks of the unprocessed blood samples associated with the plurality of cancer patients, measuring a first set of ratio of LDNs to HDNs in the unprocessed blood samples associated with the plurality of cancer patients utilizing a cell counter, and assigning each measured ratio of LDNs to HDNs of the first set of ratio of LDNs to HDNs to the respective measured electrical current peak of the first set of electrical current peaks. In an exemplary embodiment, generating the second dataset of the plurality of unprocessed blood samples associated with the plurality of healthy persons may include measuring a second set of electrical current peaks of the unprocessed blood samples associated with the plurality of healthy persons, measuring a first set of ratio of LDNs to HDNs in the unprocessed blood samples associated with the plurality of healthy persons utilizing a cell counter, and assigning each measured ratio of LDNs to HDNs of the second set of ratio of LDNs to HDNs to the respective measured electrical current peak of the second set of electrical current peaks. In an exemplary embodiment, determining the first threshold electrical current value and the second threshold electrical current value may include determining the first threshold electrical current value equal to a maximum electrical current peak among the first set of electrical current peaks and determining the second threshold electrical current value equal to a minimum electrical current peak among the second set of electrical current peaks.

In an exemplary embodiment, the first threshold electrical current value is 300 μA for an adult person. In an exemplary embodiment, the first threshold electrical current value is 100 μA for a child. In an exemplary embodiment, the second threshold electrical current value is 450 μA for an adult person. In an exemplary embodiment, the second threshold electrical current value is 300 μA for a child.

In an exemplary embodiment, the method may be conducted in less than 30 seconds. In an exemplary embodiment, the method may further include acquiring the unprocessed blood sample from the suspected person to have cancer.

In an exemplary embodiment, putting the unprocessed blood sample in contact with the sensing parts of the set of the working electrode, the counter electrode, and the reference electrode of the sensor may include putting the unprocessed blood sample in contact with three respective arrays of multi-walled carbon nanotubes (VAMWCNTs) grown on the sensing parts of the set of the working electrode, the counter electrode, and the reference electrode by dropping the unprocessed blood sample on surface of the sensor.

In an exemplary embodiment, applying the set of voltages in the sweeping range from −0.8 V to +0.8 V between the reference electrode and the working electrode may include connecting respective proximal ends of the set of the working electrode, the counter electrode, and the reference electrode of the sensor to an electrical stimulator-analyzer device and applying the set of voltages in a sweeping range from −0.8 V to +0.8 V to the sensor using the electrical stimulator-analyzer device. In an exemplary embodiment, measuring the produced set of electrical currents between the counter electrode and the working electrode versus the applied set of voltages may be done utilizing the electrical stimulator-analyzer device.

In another general aspect, the present disclosure is directed to a system for real-time detecting cancer by analyzing unprocessed blood. The system may include a sensor configured to place an unprocessed blood sample drawn from a suspected person to have cancer thereon, an electrical stimulator-analyzer device electrically connected to the sensor, and a processing unit electrically connected to the electrical stimulator-analyzer device.

In an exemplary embodiment, the sensor may include a substrate and three electrodes formed on the substrate. In an exemplary embodiment, the three electrodes may include a working electrode, a counter electrode, and a reference electrode. In an exemplary embodiment, each respective electrode may include an electrically conductive layer deposited on the substrate and an array of multi-walled carbon nanotubes (VAMWCNTs). In an exemplary embodiment, the electrically conductive layer may include a proximal end and a circular distal end. In an exemplary embodiment, the array of VAMWCNTs may be grown on the circular distal end. In an exemplary embodiment, the array of VAMWCNTs may be configured to be put in contact with the unprocessed blood sample drawn from the suspected person to have cancer.

In an exemplary embodiment, the electrical stimulator-analyzer device may be electrically connected to the sensor at the respective proximal end of each respective electrode of the three electrodes. In an exemplary embodiment, the stimulator-analyzer device may be configured to apply a set of voltages in a sweeping range from −0.8 V to +0.8 V to the sensor comprising the VAMWCNTs being in contact with the unprocessed blood sample and measure a produced set of electrical currents of the sensor responsive to the applied set of voltages.

In an exemplary embodiment, the processing unit may include a memory having processor-readable instructions stored therein and a processor. In an exemplary embodiment, the processor may be configured to access the memory and execute the processor-readable instructions. In an exemplary embodiment, when the processor-readable instructions are executed by the processor, configures the processor to perform a method. In an exemplary embodiment, the method may include applying the set of voltages in the sweeping range from −0.8 V to +0.8 V between the reference electrode and the working electrode utilizing the stimulator-analyzer device, measuring the produced set of electrical currents between the counter electrode and the working electrode utilizing the stimulator-analyzer device, measuring a level of a ratio of low-density neutrophils (LDNs) to high-density neutrophils (HDNs) in the unprocessed blood sample by measuring a level of reactive oxygen species (ROS) in the unprocessed blood sample, and detecting a cancer status of the suspected person. In an exemplary embodiment, measuring the level of ROS in the unprocessed blood sample may include measuring a maximum electrical current of the measured set of electrical currents.

In an exemplary embodiment, detecting the cancer status of the suspected person may include one of detecting a cancer disease in suspected person's body if the measured ratio of LDNs to HDNs in the unprocessed blood sample is more than 1 by detecting the measured maximum electrical current of the measured set of electrical currents being less than a first threshold electrical current value or detecting no cancer disease in the suspected person's body if the measured ratio of LDNs to HDNs in the unprocessed blood sample is less than 1 by detecting the measured maximum electrical current of the measured set of electrical currents being more than a second threshold electrical current value. In an exemplary embodiment, the method may be conducted in a time period of less than 30 seconds.

In an exemplary embodiment, detecting the cancer disease in the suspected person's body may include detecting the cancer disease in the suspected person's body if the suspected person's body is an adult and if the measured maximum electrical current of the measured set of electrical currents is less than 300 μA corresponding to a ratio of LDNs to HDNs in the unprocessed blood sample being in a range of more than 1. In another exemplary embodiment, detecting the cancer disease in the suspected person's body may include detecting the cancer disease in the suspected person's body if the suspected person's body is a child and if the measured maximum electrical current of the measured set of electrical currents is less than 100 μA corresponding to a ratio of LDNs to HDNs in the unprocessed blood sample being in a range of more than 1.

In an exemplary embodiment, the method may further include detecting no cancer disease (a normal or healthy status) in the suspected person's body if the measured ratio of LDNs to HDNs in the unprocessed blood sample is less than a threshold LDNs/HDNs value of 1. In an exemplary embodiment, detecting no cancer disease (a normal or healthy status) in the suspected person's body may include detecting no cancer disease in the suspected person's body if the suspected person's body is an adult and if the measured maximum electrical current of the measured set of electrical currents is more than 450 μA corresponding to a ratio of LDNs to HDNs in the unprocessed blood sample being less than 1. In another exemplary embodiment, detecting no cancer disease (a normal or healthy status) in the suspected person's body may include detecting no cancer disease in the suspected person's body if the suspected person's body is a child and if the measured maximum electrical current of the measured set of electrical currents is more than 300 μA corresponding to a ratio of LDNs to HDNs in the unprocessed blood sample being less than 1.

In an exemplary embodiment, the method may further include determining the first threshold electrical current value and the second threshold electrical current value. In an exemplary embodiment, determining the first threshold electrical current value and the second threshold electrical current value may include generating a first dataset of a plurality of unprocessed blood samples associated with a plurality of cancer patients, generating a second dataset of a plurality of unprocessed blood samples associated with a plurality of healthy persons, and determining the first threshold electrical current value and the second threshold electrical current value.

In an exemplary embodiment, generating the first dataset of the plurality of unprocessed blood samples associated with the plurality of cancer patients may include measuring a first set of electrical current peaks of the unprocessed blood samples associated with the plurality of cancer patients, measuring a first set of ratio of LDNs to HDNs in the unprocessed blood samples associated with the plurality of cancer patients utilizing a cell counter, and assigning each measured ratio of LDNs to HDNs of the first set of ratio of LDNs to HDNs to the respective measured electrical current peak of the first set of electrical current peaks. In an exemplary embodiment, generating the second dataset of the plurality of unprocessed blood samples associated with the plurality of healthy persons may include measuring a second set of electrical current peaks of the unprocessed blood samples associated with the plurality of healthy persons, measuring a first set of ratio of LDNs to HDNs in the unprocessed blood samples associated with the plurality of healthy persons utilizing a cell counter, and assigning each measured ratio of LDNs to HDNs of the second set of ratio of LDNs to HDNs to the respective measured electrical current peak of the second set of electrical current peaks. In an exemplary embodiment, determining the first threshold electrical current value and the second threshold electrical current value may include determining the first threshold electrical current value equal to a maximum electrical current peak among the first set of electrical current peaks and determining the second threshold electrical current value equal to a minimum electrical current peak among the second set of electrical current peaks.

In an exemplary embodiment, the circular distal end has a diameter in a range of 0.5 mm to 3 mm. In an exemplary embodiment, the circular distal end has a diameter of 2 mm. In an exemplary embodiment, the three respective circular distal ends of the three electrodes may be placed apart from each other by a distance between 1 mm and 5 mm. In an exemplary embodiment, the three respective circular distal ends of the three electrodes may be placed apart from each other by a distance of 5 mm.

In an exemplary embodiment, the substrate may include a first layer of silicon dioxide deposited on a layer of silicon. In an exemplary embodiment, the sensor may further include a second layer of silicon dioxide deposited on surface of sensor except surface of the circular distal end and the proximal end of each respective electrode of the three electrodes.

In an exemplary embodiment, the electrically conductive layer may include a layer of at least one of nickel, gold, and combinations thereof. In an exemplary embodiment, the electrically conductive layer may have a thickness in a range of 5 nm to 20 nm.

In an exemplary embodiment, the array of VAMWCNTs may include VAMWCNTs with a length in a range of 2.5 μm to 5 μm. In an exemplary embodiment, the array of VAMWCNTs may include VAMWCNTs with a diameter in a range of 50 nm to 70 nm.

### BRIEF DESCRIPTION OF THE DRAWINGS

The drawing figures depict one or more embodiments in accord with the present teachings, by way of example only, not by way of limitation. In the figures, like reference numerals refer to the same or similar elements.

FIG. 1A shows an exemplary system for real-time cancer detection, consistent with one or more exemplary embodiments of the present disclosure.

FIG. 1B shows a schematic top view of an exemplary sensor for real-time cancer detection, consistent with one or more exemplary embodiments of the present disclosure.

FIG. 1C shows a schematic view of an exemplary sensor for real-time cancer detection, consistent with one or more exemplary embodiments of the present disclosure.

FIG. 1D shows another schematic view of an exemplary sensor for real-time cancer detection, consistent with one or more exemplary embodiments of the present disclosure.

FIG. 1E shows a schematic view of an exemplary sensor with an exemplary holder and an exemplary cap, consistent with one or more exemplary embodiments of the present disclosure.

FIG. 2A shows an exemplary flowchart of an exemplary method for real-time cancer detection, consistent with one or more exemplary embodiments of the present disclosure.

FIG. 2B shows an exemplary flowchart of another exemplary method for real-time cancer detection, consistent with one or more exemplary embodiments of the present disclosure.

FIG. 2C shows a flowchart of an exemplary method for generating an exemplary first threshold electrical current value and an exemplary second threshold electrical current value, consistent with one or more exemplary embodiments of the present disclosure.

FIG. 3A shows an exemplary flowchart of an exemplary method for fabricating an exemplary sensor, consistent with one or more exemplary embodiments of the present disclosure.

FIG. 3B shows a schematic view of steps of an exemplary method for fabricating an exemplary sensor, consistent with one or more exemplary embodiments of the present disclosure.

FIG. 4 shows comparative cyclic voltammograms of an exemplary cancer patient and an exemplary healthy candidate, consistent with one or more exemplary embodiments of the present disclosure.

FIG. 5 shows an exemplary computer system in which an embodiment of the present disclosure, or portions thereof, may be implemented as computer-readable code, consistent with one or more exemplary embodiments of the present disclosure.

FIG. 6 shows an image of a Field Emission Scanning Electron Microscope (FE-SEM) of exemplary grown CNTs, consistent with one or more exemplary embodiments of the present disclosure.

FIG. 7 shows a chart representing repeatability of an exemplary fabricated sensor obtained by testing three blood samples from normal and cancer patients with a 3-times of test repeat for each blood sample, consistent with one or more exemplary embodiments of the present disclosure.

FIG. 8 shows a chart of measured peak currents associated with ROS levels in blood samples of 160 investigated patients for two groups of G1: normal candidate/patients with non-cancer diseases and G2: patients with newborns cancer, consistent with one or more exemplary embodiments of the present disclosure.

FIG. 9 shows a receiver operating characteristic (ROC) diagram for results obtained by utilizing exemplary method and fabricated sensor versus clinical diagnostics results for blood samples from 160 patients, consistent with one or more exemplary embodiments of the present disclosure.

FIG. 10A shows representative images of ROS/H₂O₂ related electrochemical current peak, optical microscope images of LDNs and HDNs, fluorescent microscope images of LDNs and HDNs, Giemsa staining images of LDNs and HDNs, and total cell count of LDNs and HDNs isolated by magnetic, negative selection protocol neutrophils isolated from 5 ml blood of normal cases (N=10), consistent with one or more exemplary embodiments of the present disclosure.

FIG. 10B shows representative images of ROS/H₂O₂ related electrochemical current peak, optical microscope images of LDNs and HDNs, fluorescent microscope images of LDNs and HDNs, Giemsa staining images of LDNs and HDNs, and total cell count of LDNs and HDNs isolated by magnetic, negative selection protocol neutrophils isolated from 5 ml blood of cancer patients (N=10), consistent with one or more exemplary embodiments of the present disclosure.

FIG. 10C shows a chart of LDN/HDN ratio versus measured current peak diagram with a fitted plot for 20 cases, including normal cases (N=10) and cancer patients (N=10), consistent with one or more exemplary embodiments of the present disclosure.

FIG. 11 shows a chart of ROS/H₂O₂ assisted electrochemical current peaks of blood samples of patients with non-cancer diseases (n=15), consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings. The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary embodiments will be readily apparent to one skilled in the art, and the general principles defined herein may be applied to other embodiments and applications without departing from the scope of the present disclosure. The present disclosure is not intended to be limited to the embodiments shown, but is to be accorded the widest possible scope consistent with the principles and features disclosed herein.

In cancer patients with solid tumors, neutrophils can paradoxically mediate a broad range of anti- and pro-tumor activities from cancer cell killing (done by HDNs) to tumor cell proliferation, angiogenesis, metastasis, and organizing other immune responses (done by LDNs). Neutrophil density changes due to their increased granularity and variations in cell size during development. Immature neutrophils are typically found in low density (LD) fractions, whereas mature neutrophils are detected in normal/high density (N/HD) fractions. LDNs are either immature with a banded/ring nucleus or mature with segmented nuclei. All LDNs are pro-tumor that present immunosuppressive properties. HDNs (or NDNs) are mature with segmented nuclei which are antitumor. It should be noted that TGF-β could also mediate transition of HDN to LDN (displaying plasticity) in the murine model.

An increased number of immature neutrophils in peripheral blood and tissues is a consequence of cancer development in human patients. LDNs may be detectable in peripheral blood of patients with lung cancer, breast cancer, and ovarian cancer. This increase may result from promotion of immature neutrophil production and release due to increased systemic chemokines, (e.g., granulocyte colony-stimulating (G-CSF)). It should be noted that tumors produce granulocyte colony-stimulating factor (G-CSF). G-CSF reduces chemokine receptor type 4 (CXCR4) expression in human myeloid lineage cells, resulting in decreasing their response to the bone marrow retention signal stromal cell-derived factor 1 (SDF-1).

Immature neutrophils may have both anti- and pro-tumor properties. These include altered localization resulting from reduced surface chemotactic receptor expression (e.g., CXCR2) and less segmented nuclear morphology compared to mature ones which reduce their immunological functions due to their reduced ROS production and altered cell surface receptor expression. Also, immature neutrophils are unable to kill tumor cell via FcγRI receptors. These differences in properties and functions of immature neutrophils could lead to their negative effect on cancer therapy. ROS production is crucial in several neutrophil effector mechanisms including their microbicidal, phagocytic, suppressive capacity and neutrophil anti, pro-tumor functions. Reduced ROS production may be the main deficiency of immature neutrophils against cancer cells.

Herein, an exemplary real-time electrochemical diagnostic sensor, system, and method for detecting cancer by detecting a level of a ratio of LDNs/HDNs in a blood sample via detecting reactive oxygen species (ROS) levels in a small amount (i.e., about 1 cc) of peripheral blood is disclosed. An exemplary sensor may include three disks like electrodes (working electrode (WE), counter electrode (CE), and reference electrode (RE)) covered by Multi-Wall Carbon Nanotubes (MWCNTs) as a ROS detecting agent in a solution. A cancerous status of a suspected person may be determined utilizing exemplary sensor, system, and/or method in a time period of less than about 30 seconds. An exemplary method and system may be utilized for a rapid detection of cancer via a simple procedure using a small amount (i.e., about 1 cc) of an unprocessed blood sample drawn from a person suspected to have cancer.

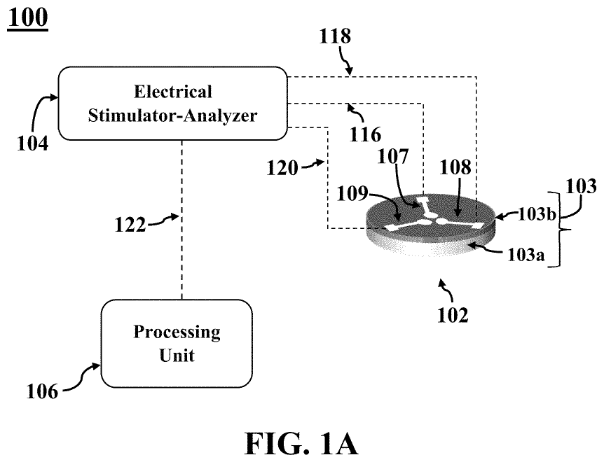

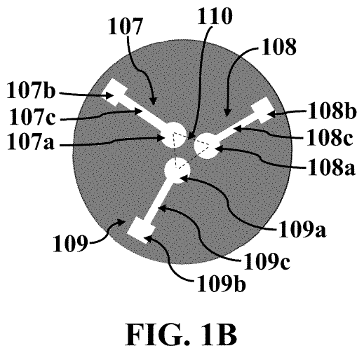

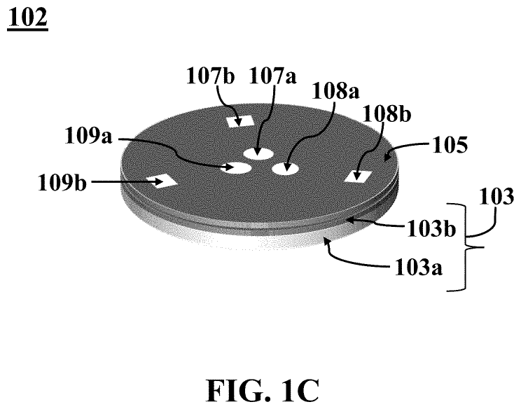

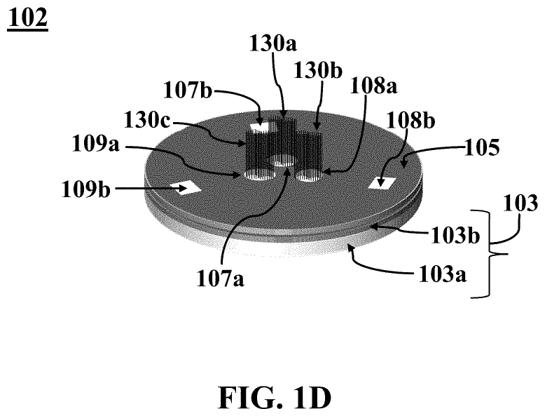

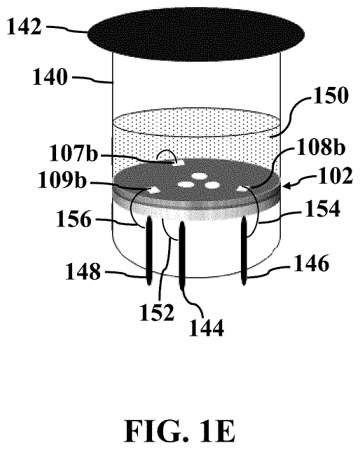

FIG. 1A shows an exemplary system 100 for real-time cancer detection, consistent with one or more exemplary embodiments of the present disclosure. In an exemplary embodiment, system 100 may include an exemplary sensor 102, an exemplary electrical stimulator-analyzer device 104, and an exemplary processing unit 106. In an exemplary embodiment, sensor 102 may be configured to receive a biological sample thereon. In an exemplary embodiment, the biological sample may be acquired from a person suspected to have cancer. In an exemplary embodiment, the biological sample may include a blood sample drawn from a person. In an exemplary embodiment, the biological sample may include an unprocessed blood sample drawn from a person and directly placed on surface of sensor 102. In an exemplary embodiment, one or more drops of an unprocessed blood sample may be dropped on surface of sensor 102. In an exemplary embodiment, sensor 102 may be electrically connected to electrical stimulator-analyzer device 104 and electrical stimulator-analyzer device 104 may be electrically connected to processing unit 106.

In an exemplary embodiment, sensor 102 may include an exemplary electrochemical sensor. In an exemplary embodiment, sensor 102 may include substrate 103 and three electrodes 107, 108, and 109 formed on substrate 103. In an exemplary embodiment, three electrodes 107, 108, and 109 may include a set of electrochemical electrodes configured to be utilized for electrochemical measurements. In an exemplary embodiment, three electrodes 107, 108, and 109 may include a working electrode 107, a counter electrode 108, and a reference electrode 109. FIG. 1B shows a schematic top view of an exemplary sensor 102 for real-time cancer detection, consistent with one or more exemplary embodiments of the present disclosure. In an exemplary embodiment, each respective electrode of three electrodes 107, 108, and 109 may include a circular sensing part, a connecting pad, and a middle part between the circular sensing part and the connecting pad. In detail, referring to FIG. 1B, an exemplary working electrode 107 may include an exemplary circular sensing part 107a, an exemplary connecting pad 107b, and an exemplary middle part 107c. Furthermore, an exemplary counter electrode 108 may include an exemplary circular sensing part 108a, an exemplary connecting pad 108b, and an exemplary middle part 108c. Moreover, an exemplary reference electrode 109 may include an exemplary circular sensing part 109a, an exemplary connecting pad 109b, and an exemplary middle part 109c. In an exemplary embodiment, three electrodes 107, 108, and 109 may be made of an electrically conductive material. In an exemplary embodiment, three electrodes 107, 108, and 109 may be made of a metal. In an exemplary embodiment, three electrodes 107, 108, and 109 may be made of at least one of nickel, gold, and combinations thereof. In an exemplary embodiment, circular sensing parts 107a, 108a, and 109a may include three respective circular distal ends of three respective electrodes 107, 108, and 109. In an exemplary embodiment, circular sensing parts 107a, 108a, and 109a may be configured to be put in contact with an exemplary unprocessed blood sample dropped on surface of exemplary sensor 102. In an exemplary embodiment, connecting pads 107b, 108b, and 109b may include three respective proximal ends of three respective electrodes 107, 108, and 109. In an exemplary embodiment, connecting pads 107b, 108b, and 109b may be configured to be electrically connected to electrical stimulator-analyzer device 104 via respective electrically conductive lines 116, 118, and 120 as shown in FIG. 1A.

In an exemplary embodiment, each respective electrode of three electrodes 107, 108, and 109 may include an electrically conductive layer deposited on substrate 103. In an exemplary embodiment, an exemplary electrically conductive layer may have a thickness in a range of about 5 nm to about 20 nm. In an exemplary embodiment, an exemplary electrically conductive layer may include a catalyst material for a process of growing carbon nanotubes (CNTs), for example, vertically aligned multi-walled CNTs (VAMWCNTs), thereon. In an exemplary embodiment, an exemplary electrically conductive layer may include a layer of at least one of nickel, gold, and combinations thereof. In an exemplary embodiment, an exemplary electrically conductive layer may include a circular distal end and a proximal end. In an exemplary embodiment, an exemplary circular distal end may form an exemplary circular sensing part similar to circular sensing parts 107a, 108a, and 109a and an exemplary proximal end may form an exemplary connecting pad similar to connecting pads 107b, 108b, and 109b. In an exemplary embodiment, each respective circular sensing part of circular sensing parts 107a, 108a, and 109a may have a diameter in a range of about 0.5 mm to about 3 mm. In an exemplary embodiment, each respective circular sensing part of circular sensing parts 107a, 108a, and 109a may have a diameter of about 2 mm. In an exemplary embodiment, circular sensing parts 107a, 108a, and 109a of three respective electrodes 107, 108, and 109 may be placed on substrate 103 apart from each other by a distance between about 1 mm and about 5 mm. In an exemplary embodiment, circular sensing parts 107a, 108a, and 109a of three respective electrodes 107, 108, and 109 may be placed on substrate 103 apart from each other by a triangular distance 110 (illustrated in FIG. 1B) between of about 5 mm.

Referring back to FIG. 1A, substrate 103 may include a semi-conductive layer 103a and a first electrically passivating layer 103b deposited on semi-conductive layer 103a. In an exemplary embodiment, semi-conductive layer 103a may include a layer of silicon. In an exemplary embodiment, first electrically passivating layer 103b may include a first layer of silicon dioxide (SiO₂). In an exemplary embodiment, three electrodes 107, 108, and 109 may include a layer of an electrically conductive material deposited and patterned on first electrically passivating layer 103b.

In an exemplary embodiment, sensor 102 may further include a second electrically passivating layer deposited on surface of sensor 102 except surface of circular sensing parts 107a, 108a, and 109a and surface of connecting pads 107b, 108b, and 109b of three respective electrodes 107, 108, and 109. FIG. 1C shows a schematic view of an exemplary sensor 102 for real-time cancer detection, consistent with one or more exemplary embodiments of the present disclosure. In an exemplary embodiment with reference to FIG. 1C, exemplary sensor 102 may further include a second electrically passivating layer 105 deposited on substrate 103. In an exemplary embodiment, second electrically passivating layer 103b may include a second layer of SiO₂ deposited on first electrically passivating layer 103b and three middle parts 107c, 108c, and 109c of three respective electrodes 107, 108, and 109.

In an exemplary embodiment, each respective circular sensing part of circular sensing parts 107a, 108a, and 109a may be coated with a layer of carbon nanotubes (CNTs) deposited on the respective circular sensing part. FIG. 1D shows another schematic view of exemplary sensor 102 for real-time cancer detection, consistent with one or more exemplary embodiments of the present disclosure. In an exemplary embodiment, three arrays of CNTs 130a, 130b, and 130c may be coated on respective circular sensing parts 107a, 108a, and 109a. In an exemplary embodiment, arrays of CNTs 130a, 130b, and 130c may include three respective arrays of vertically aligned multi-walled carbon nanotubes (VAMWCNTs) grown on respective circular sensing parts 107a, 108a, and 109a. In an exemplary embodiment, circular sensing parts 107a, 108a, and 109a may be configured to be put in contact with an exemplary unprocessed blood sample dropped on surface of exemplary sensor 102. In an exemplary embodiment, three arrays of CNTs 130a, 130b, and 130c may be configured to be put in contact with an exemplary unprocessed blood sample dropped on surface of exemplary sensor 102. In an exemplary embodiment, each respective array of CNTs of three arrays of CNTs 130a, 130b, and 130c may include a respective plurality of VAMWCNTs, where each respective VAMWCNT may have a length in a range of about 2.5 μm to about 5 μm. In an exemplary embodiment, each respective VAMWCNT of an exemplary array of VAMWCNTs may have a diameter in a range of about 50 nm to about 70 nm.

In an exemplary embodiment, system 100 may further include a holder and a cap. FIG. 1E shows a schematic view of exemplary sensor 102 with a holder 140 and a cap 142, consistent with one or more exemplary embodiments of the present disclosure. In an exemplary embodiment, exemplary holder 140 may include a hollow cylinder with a diameter equal or more than an outer diameter of sensor 102. In an exemplary embodiment, exemplary holder 140 may be configured to fix sensor 102 with an exemplary unprocessed blood sample 150 therein, allowing for protecting sensor 102 and unprocessed blood sample 150 while being in contact with each other. In an exemplary embodiment, exemplary holder 140 may include three electrically conductive elements (pins) 144, 146, and 148, where each respective pin may be in contact with a respective connecting pad of connecting pads 107b, 108b, and 109b of sensor 102, allowing for an electrical connection between three electrodes 107, 108, and 109 of sensor 102 and three pins 144, 146, and 148. In an exemplary embodiment, three pins 144, 146, and 148 may be configured to connect respective electrodes 107, 108, and 109 of sensor 102 to an exemplary electrical stimulator-analyzer device 104. In an exemplary embodiment, sensor 102 may be placed inside holder 140 so that each respective pin of three pins 144, 146, and 148 being in contact with a respective electrode of three electrodes 107, 108, and 109 of sensor 102. In an exemplary embodiment, unprocessed blood sample 150 may be dropped inside holder 140 on surface of sensor 102.

In an exemplary embodiment, an exemplary cap 142 may include a circular element with a diameter equal or more than an outer diameter of holder 140. In an exemplary embodiment, cap 142 may be placed on a top side of holder 140 above sensor 102 and unprocessed blood sample 150. In an exemplary embodiment, cap 142 may be configured to protect sensor 102 and unprocessed blood sample 150 being in contact with each other away from pollutants and disturbing factors.

In an exemplary embodiment, electrical stimulator-analyzer device 104 may be electrically connected to sensor 102 at respective proximal ends of each respective electrode of three electrodes 107, 108, and 109 of sensor 102. In an exemplary embodiment, electrical stimulator-analyzer device 104 may be electrically connected to sensor 102 at connecting pads 107b, 108b, and 109b of three electrodes 107, 108, and 109 utilizing three respective electrically conductive lines 116, 118, and 120. In an exemplary embodiment, electrical stimulator-analyzer device 104 may include a potentiostat device. In an exemplary embodiment, electrical stimulator-analyzer device 104 may be configured to apply a set of voltages in a sweeping range from about −3 V to about +3 V, for example, a range of −0.8 V to +0.8 V, to sensor 102 with unprocessed blood sample 150 placed thereon and measure a produced set of electrical currents of sensor 102 responsive to the applied set of voltages. In an exemplary embodiment, electrical stimulator-analyzer device 104 may be electrically connected to processing unit 106 utilizing at least one of an electrically conductive line 122, a wireless connection (not illustrated), and combinations thereof. In an exemplary embodiment, the wireless connection may include Bluetooth devices or Bluetooth modules, which may be embedded in electrical stimulator-analyzer device 104 and processing unit 106. In an exemplary embodiment, electrical stimulator-analyzer device 104 may be further configured to send the measured set of electrical currents to processing unit 106.

In an exemplary embodiment, processing unit 106 may include a memory having processor-readable instructions stored therein and a processor. In an exemplary embodiment, the processor may be configured to access the memory and execute the processor-readable instructions. In an exemplary embodiment, executing the processor-readable instructions by the processor may configures the processor to perform a method. In an exemplary embodiment, the method may include an exemplary method for real-time detecting cancer described herein below.

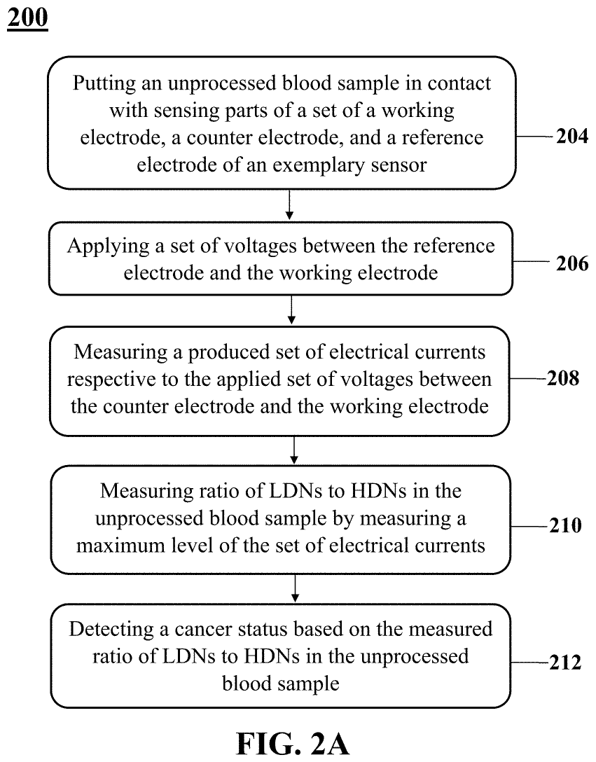

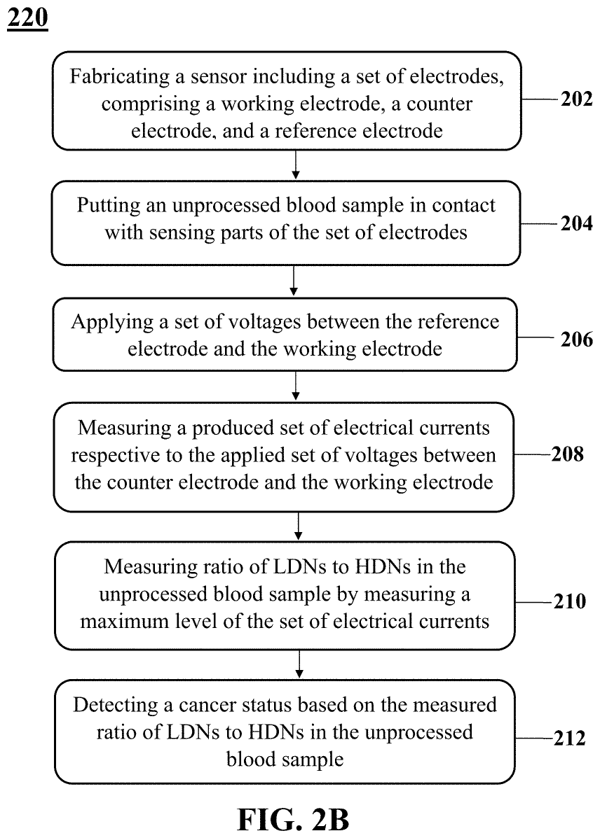

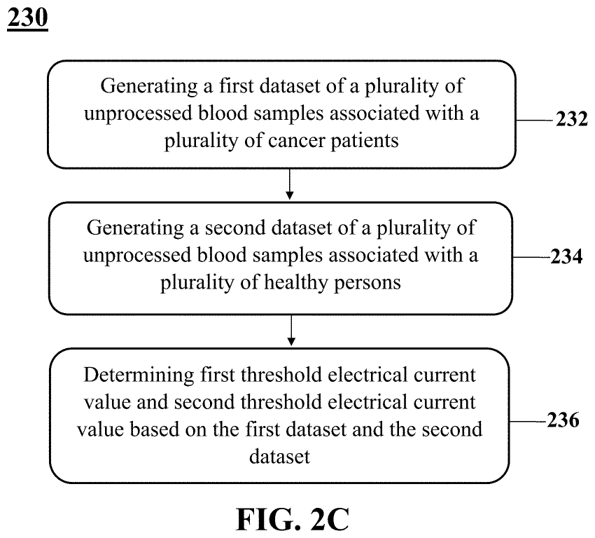

FIG. 2A shows a flowchart of an exemplary method 200 for real-time cancer detection, consistent with one or more exemplary embodiments of the present disclosure. In an exemplary embodiment, method 200 may include putting an unprocessed blood sample in contact with sensing parts of a set of electrodes of a sensor (step 204), applying a set of voltages to the sensor (step 206), measuring a produced set of electrical currents respective to the applied set of voltages (step 208), measuring ratio of LDNs to HDNs in the unprocessed blood sample by measuring a maximum level of the set of electrical currents (step 210), and detecting a cancer status based on the measured ratio of LDNs to HDNs in the unprocessed blood sample (step 212).

FIG. 2B shows a flowchart of another exemplary method 220 for real-time detecting cancer, consistent with one or more exemplary embodiments of the present disclosure. In an exemplary embodiment, exemplary method 220 may contain steps similar to exemplary method 200 while further including step 202 of fabricating an exemplary sensor. Steps of exemplary method 200 and 220 are explained below in further detail in combination with the elements of FIGS. 1A-1E.

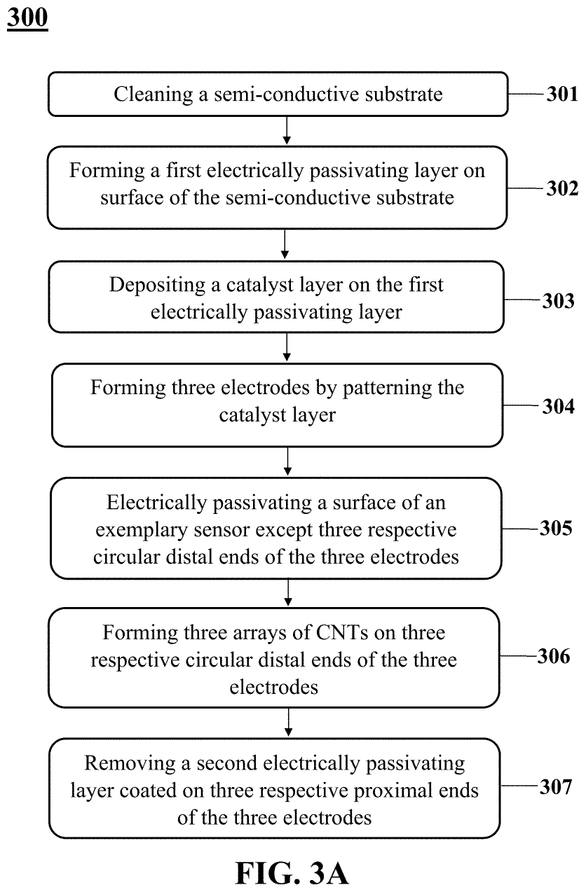

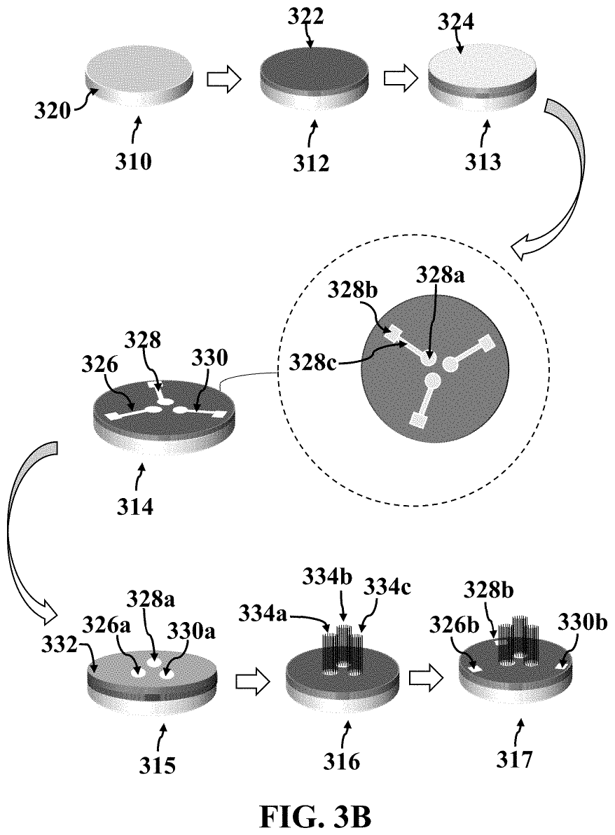

With further detail in regards to step 202, FIG. 3A shows a flowchart of an exemplary method 300 for fabricating an exemplary sensor (step 202), consistent with one or more exemplary embodiments of the present disclosure. In an exemplary embodiment, exemplary method 300 of fabricating an exemplary sensor (step 202) may include cleaning a semi-conductive substrate (step 301), forming a first electrically passivating layer on surface of the semi-conductive substrate (step 302), depositing a catalyst layer on the first electrically passivating layer (step 303), forming three electrodes by patterning the catalyst layer (step 304), electrically passivating a surface of an exemplary sensor except three respective circular distal ends of the three electrodes by coating a second electrically passivating layer on surface of an exemplary sensor except the three respective circular distal ends of the three electrodes (step 305), forming three arrays of CNTs on three respective circular distal ends of the three electrodes (step 306), and removing parts of the second electrically passivating layer coated on three respective proximal ends of the three electrodes (step 307). FIG. 3B shows a schematic view of steps of exemplary method 300 for fabricating an exemplary sensor, consistent with one or more exemplary embodiments of the present disclosure. In an exemplary embodiment, an exemplary sensor may be similar to sensor 102, and exemplary method 300 may be described herein below in connection with FIG. 3B and FIGS. 1A-1E illustrating an exemplary structure of exemplary sensor 102.

With further detail in regards to step 301, an exemplary semi-conductive substrate may be cleaned. In an exemplary embodiment, an exemplary semi-conductive substrate may include semi-conductive layer 310 shown in part 310 of FIG. 3B (similar to semi-conductive layer 103a of FIGS. 1A, 1C, and 1D). In an exemplary embodiment, exemplary semi-conductive layer 320 may include an exemplary circular silicon (Si) wafer. In an exemplary embodiment, semi-conductive layer 320 may include a P-type Si wafer. In an exemplary embodiment, cleaning semi-conductive layer 320 (step 301) may include cleaning semi-conductive layer 320 allowing for removing contaminations from an exemplary silicon wafer using a standard RCA method.

In further detail with respect to step 302, step 302 may include forming a first electrically passivating layer on surface of an exemplary cleaned semi-conductive substrate. In an exemplary embodiment, an exemplary first electrically passivating layer 322 (similar to first electrically passivating layer 103b of FIGS. 1A, 1C, and 1D) may be coated on surface of semi-conductive layer 320 in step 302 as shown in part 312 of FIG. 3B; thereby, an exemplary substrate similar to substrate 103 may be formed. In an exemplary embodiment, step 302 may include growing a first silicon dioxide (SiO₂) layer as an example of first electrically passivating layer 322 on a cleaned silicon wafer as an example of semi-conductive layer 320 cleaned in step 301.

In further detail with respect to step 303, step 303 may include depositing a catalyst layer on an exemplary first electrically passivating layer as schematically shown in part 313 of FIG. 3B. In an exemplary embodiment, step 303 may include depositing a catalyst layer 324 on exemplary first electrically passivating layer 322. In an exemplary embodiment depositing catalyst layer 324 on an exemplary first electrically passivating layer 322 (step 303) may include depositing a layer of a catalyst to be used for conducting a process of growing CNTs on exemplary first electrically passivating layer 322. In an exemplary embodiment, catalyst layer 324 may include a layer of an electrically conductive catalyst for a process of growing CNTs thereon. In an exemplary embodiment, depositing exemplary catalyst layer 324 on exemplary first electrically passivating layer 322 (step 303) may include depositing a layer of nickel (Ni) on an exemplary first silicon dioxide (SiO₂) layer as an example of exemplary first electrically passivating layer 322 formed on an exemplary cleaned silicon wafer as an example of semi-conductive layer 320 obtained via steps 301 and 302. In an exemplary embodiment, exemplary catalyst layer 324 may have a thickness of less than about 20 nm. In an exemplary embodiment, exemplary catalyst layer 324 may have a thickness of about 10 nm or less.

In further detail with respect to step 304, step 304 may include forming three exemplary electrodes 326, 328, and 330 (similar to exemplary three exemplary electrodes 107, 108, and 109 of FIGS. 1A-1B) by patterning exemplary catalyst layer 324 as schematically shown in part 314 of FIG. 3B. In an exemplary embodiment, three exemplary electrodes 326, 328, and 330 may form a set of electrodes including an exemplary working electrode 326, an exemplary counter electrode 328, and an exemplary reference electrode 330. In an exemplary embodiment, patterning exemplary catalyst layer 324 may be done via a photolithography technique. In an exemplary embodiment, patterning exemplary catalyst layer 324 may include patterning exemplary catalyst layer 324 in form of three exemplary electrodes 326, 328, and 330. In an exemplary embodiment, patterning exemplary catalyst layer 324 may include patterning exemplary catalyst layer 324 in shape of three similar structures with circular heads (for example, circular head 328a of electrode 328) with a diameter of about 2 mm in conformation of three electrodes 326, 328, and 330, as working, counter, and reference electrodes with a triangular distance of about 5 mm from each other using a standard photolithography process. In an exemplary embodiment, patterning exemplary catalyst layer 324 may further include spin-coating a thin layer of a positive photoresist (e.g., Microchem-S1813) on surface of exemplary catalyst layer 324, chemically developing semi-conductive layer 320 with first electrically passivating layer 322 and exemplary catalyst layer 324 thereon by illuminating with a Mask-Aligner system, etching parts of exemplary catalyst layer 324 in undesired regions using an etchant solution (e.g., a Ni-etchant solution: HNO₃:H₃PO₄:CH₃COOH, 3:3:1), and washing the photoresist layer using a solvent, for example, acetone. In an exemplary embodiment, exemplary undesired regions may include parts of exemplary catalyst layer 324 around patterned three exemplary electrodes 326, 328, and 330.

In an exemplary embodiment, each exemplary electrode 328 of three exemplary electrodes 326, 328, and 330 may include a respective circular head 328a (similar to each of circular sensing parts 107a, 108a, and 109a illustrated in FIGS. 1B-1D). Moreover, each exemplary electrode 328 of three exemplary electrodes 326, 328, and 330 may include a respective tail 328b similar to each of connecting pads 107b, 108b, and 109b illustrated in FIGS. 1B-1D. In an exemplary embodiment, exemplary circular head 328a may include a respective distal end of exemplary electrode 328 of set of electrodes 326, 328, and 330. In an exemplary embodiment, exemplary tail 328 may include a respective proximal end of exemplary electrode 328 of set of electrodes 326, 328, and 330. In an exemplary embodiment, each respective electrode of set of electrodes 326, 328, and 330, for example, exemplary electrode 328 may further include a respective middle part 328c between respective circular head 328a and respective tail 328b. In an exemplary embodiment, respective circular heads (e.g., circular head 328a) of electrodes 326, 328, and 330 may be configured to be put in contact with a sample (e.g., exemplary unprocessed blood sample 150) by placing an exemplary sample thereon. In an exemplary embodiment, respective tails (e.g., tail 328b) of electrodes 326, 328, and 330 may be configured to be connected to an electrical device, for example, electrical stimulator-analyzer device 104. In an exemplary embodiment, respective middle parts (e.g., middle part 328c) of electrodes 326, 328, and 330 may be configured to form an electrically conductive path between respective circular heads (e.g., circular head 328a) of electrodes 326, 328, and 330, and similarly, circular sensing parts 107a, 108a, and 109a and respective tails (e.g., tail 328b) of electrodes 326, 328, and 330, and similarly, connecting pads 107b, 108b, and 109b of electrodes 107, 108, and 109.

In further detail with respect to step 305, step 305 may include electrically passivating a surface of an exemplary sensor except three respective circular distal ends of exemplary three electrodes by coating a second electrically passivating layer on surface of an exemplary sensor except three respective circular distal ends of three exemplary electrodes. In an exemplary embodiment, step 305 may include electrically passivating a surface of exemplary sensor 102 except three respective circular heads 326a, 328a, and 330a) of electrodes 326, 328, and 330 as schematically shown in part 315 of FIG. 3B. In an exemplary embodiment, step 305 may include coating entire surface of exemplary sensor 102 with a second electrically passivating layer 332 (similar to second electrically passivating layer 105) and removing parts of exemplary second electrically passivating layer 332 coated on circular heads 326a, 328a, and 330a of respective electrodes 326, 328, and 330. In an exemplary embodiment, entire surface of exemplary sensor 102 may include surface of first electrically passivating layer 322 and surface of patterned electrodes 326, 328, and 330. In an exemplary embodiment, second electrically passivating layer 332 may include a second SiO₂ layer. In an exemplary embodiment, coating entire surface of exemplary sensor 102 with second electrically passivating layer 332 may include passivating exemplary sensor 102 with a 200 nm SiO₂ layer at 0.9 torr, and 20 W (deposition speed <45 nm min⁻¹). In an exemplary embodiment, removing parts of an exemplary second SiO₂ layer coated on respective circular heads 326a, 328a, and 330a of electrodes 326, 328, and 330 may be carried out by immersing exemplary sensor 102 in a buffered oxide etchant (BOE) solution to prepare circular windows for three electrodes 326, 328, and 330. In an exemplary embodiment, immersing exemplary sensor 102 in the BOE solution may be done in about 7 seconds.

In further detail with respect to step 306, step 306 may include forming three arrays of CNTs on three respective circular distal ends of three exemplary electrodes of an exemplary sensor as schematically shown in part 316 of FIG. 3B. In an exemplary embodiment, step 306 may include forming three exemplary arrays of CNTs 334a, 334b, and 334c (similar to three exemplary arrays of CNTs 130a, 130b, and 130c of FIG. 1D) on three respective circular heads 326a, 328a, and 330a of electrodes 326, 328, and 330. In an exemplary embodiment, forming three arrays of CNTs 334a, 334b, and 334c on respective circular heads 326a, 328a, and 330a of electrodes 326, 328, and 330 may include growing a respective plurality of vertically aligned multi-walled carbon nanotubes (VAMWCNTs) on each respective circular head of circular heads 326a, 328a, and 330a of electrodes 326, 328, and 330. In an exemplary embodiment, growing VAMWCNTs may be carried out in a direct current plasma enhance chemical vapor deposition (DC-PECVD) system in C₂H₂ and H₂ ambient at a temperature of about 680° C.

In further detail with respect to step 307, step 307 may include removing parts of an exemplary second electrically passivating layer coated on three respective proximal ends of exemplary three electrodes of an exemplary sensor as schematically shown in part 317 of FIG. 3B. In an exemplary embodiment, step 307 may include removing parts of second electrically passivating layer 332 from surface of three respective proximal ends (tails 326b, 328b, and 330b) of three electrodes 326, 328, and 330. In an exemplary embodiment, step 307 may include removing parts of an exemplary coated second SiO₂ layer from surface of tails 326b, 328b, and 330b (similar to connecting pads 107b, 108b, and 109b of electrodes 107, 108, and 109) using a BOE solution.

Referring back to FIGS. 2A and 2B, step 204 may include putting an unprocessed blood sample in contact with sensing parts of electrodes of an exemplary sensor. In an exemplary embodiment, step 204 may include placing or dropping exemplary unprocessed blood sample 150 on surface of exemplary sensor 102. In an exemplary embodiment, step 204 may include putting exemplary unprocessed blood sample 150 in contact with circular sensing parts 107a, 108a, and 109a of three electrodes 107, 108, and 109 of sensor 102. In an exemplary embodiment, step 204 may include dropping exemplary unprocessed blood sample 150 on respective circular sensing parts 107a, 108a, and 109a of electrodes 107, 108, and 109. In an exemplary embodiment, exemplary unprocessed blood sample 150 may be drawn from a person suspected to have cancer. In an exemplary embodiment, exemplary unprocessed blood sample 150 may be drawn from a person who is not pregnant and/or does not infected with an inflammatory disease. In an exemplary embodiment, exemplary unprocessed blood sample 150 may be drawn from a person suspected to be involved with a cancer having a solid tumor, for example, breast cancer, colon cancer, skin cancer, etc.

In further detail with respect to step 206, step 206 may include applying a set of voltages to sensor 102. In an exemplary embodiment, applying the set of voltages to sensor 102 may include applying the set of voltages between exemplary reference electrode 109 and exemplary working electrode 107 of electrodes 107, 108, and 109 of sensor 102. In an exemplary embodiment, applying the set of voltages to sensor 102 may be done utilizing electrical stimulator-analyzer device 104. In an exemplary embodiment, applying the set of voltages to sensor 102 may include applying the set of voltages in a sweeping range from about −3 V to about +3 V between exemplary reference electrode 109 and exemplary working electrode 107 of electrodes 107, 108, and 109 of sensor 102. In an exemplary embodiment, applying the set of voltages to sensor 102 may include applying the set of voltages in the sweeping range from about −0.8 V to about +0.8 V between exemplary reference electrode 109 and exemplary working electrode 107 of electrodes 107, 108, and 109 of sensor 102.

In further detail with respect to step 208, step 208 may include measuring a produced set of electrical currents respective to the applied set of voltages to sensor 102. In an exemplary embodiment, a set of electrical currents may be generated between an exemplary counter electrode 108 and an exemplary working electrode 107 of the three electrodes 107, 108, and 109 of sensor 102 responsive to the applied set of voltages. In an exemplary embodiment, step 208 may include measuring the produced set of electrical currents between counter electrode 108 and working electrode 107 of three electrodes 107, 108, and 109 of sensor 102 respective to the applied set of voltages. In an exemplary embodiment, measuring the produced set of electrical currents may be carried out utilizing electrical stimulator-analyzer device 104. In an exemplary embodiment, measuring the produced set of electrical currents may further include sending the measured produced set of electrical currents to processing unit 106 by electrical stimulator-analyzer device 104.

In further detail with respect to step 210, step 210 may include measuring ratio of LDNs to HDNs in exemplary unprocessed blood sample 150 by measuring a maximum level of the set of electrical currents. In an exemplary embodiment, measuring the maximum level of the set of electrical currents may be carried out by one or more processors similar, for example, processing unit 106. In an exemplary embodiment, measuring the ratio of LDNs to HDNs in exemplary unprocessed blood sample 150 may include measuring a level of reactive oxygen species (ROS) in the unprocessed blood sample 150 by measuring a maximum electrical current of the measured set of electrical currents. In an exemplary embodiment, measuring the ratio of LDNs to HDNs in exemplary unprocessed blood sample 150 may include receiving the measured produced set of electrical currents, plotting a cyclic voltammetry (CV) diagram by plotting the measured produced set of electrical currents versus the applied set of voltages, and measuring a peak value of electrical currents of the CV diagram.

In an exemplary embodiment, measuring the ratio of LDNs to HDNs in exemplary unprocessed blood sample 150 may further include determining a level of ratio of LDNs to HDNs in the unprocessed blood sample using the measured maximum electrical current of the measured set of electrical currents. In an exemplary embodiment, determining the level of ratio of LDNs to HDNs in the unprocessed blood sample may include detecting the level of ratio of LDNs to HDNs in the unprocessed blood sample is more than 1 if the measured maximum electrical current of the measured set of electrical currents is less than a first threshold electrical current value and detecting the level of ratio of LDNs to HDNs in the unprocessed blood sample is less than 1 if the measured maximum electrical current of the measured set of electrical currents is more than a second threshold electrical current value.

In an exemplary embodiment, exemplary method 200 may further include generating the first threshold electrical current value and the second threshold electrical current value. FIG. 2C shows a flowchart of a method 230 for generating the first threshold electrical current value and the second threshold electrical current value, consistent with one or more exemplary embodiments of the present disclosure. In an exemplary embodiment, exemplary method 230 of generating the first threshold electrical current value and the second threshold electrical current value may include generating a first dataset of a plurality of unprocessed blood samples associated with a plurality of cancer patients (step 232), generating a second dataset of a plurality of unprocessed blood samples associated with a plurality of healthy persons (step 234), and determining first threshold electrical current value and the second threshold electrical current value based on the first dataset and the second dataset (step 236).

In further detail with respect to step 232, step 232 of generating a first dataset of a plurality of unprocessed blood samples associated with a plurality of cancer patients may include measuring a first set of electrical current peaks of the plurality of unprocessed blood samples associated with the plurality of cancer patients, measuring a first set of ratio of LDNs to HDNs in the plurality of unprocessed blood samples associated with the plurality of cancer patients utilizing a cell counter, and assigning each measured ratio of LDNs to HDNs of the first set of ratio of LDNs to HDNs to the respective measured electrical current peak of the first set of electrical current peaks.

In further detail with respect to step 234, step 234 of generating a second dataset of a plurality of unprocessed blood samples associated with a plurality of healthy persons may include measuring a second set of electrical current peaks of the plurality of unprocessed blood samples associated with the plurality of healthy persons, measuring a second set of ratio of LDNs to HDNs in the unprocessed blood samples associated with the plurality of healthy persons utilizing a cell counter, and assigning each measured ratio of LDNs to HDNs of the second set of ratio of LDNs to HDNs to the respective measured electrical current peak of the second set of electrical current peaks.

In further detail with respect to step 236, step 236 of determining the first threshold electrical current value and the second threshold electrical current value based on the first dataset and the second dataset may include determining the first threshold electrical current value equal to a maximum electrical current peak among the first set of electrical current peaks and determining the second threshold electrical current value equal to a minimum electrical current peak among the second set of electrical current peaks. In an exemplary embodiment, the first threshold electrical current value and the second threshold electrical current value may be equal to each other.

In an exemplary embodiment, the first threshold electrical current value and the second threshold electrical current value may depend on age of an exemplary suspected person. In an exemplary embodiment, the first threshold electrical current value may be equal to an electrical current peak of about 300 μA for an adult person. In an exemplary embodiment, the first threshold electrical current value may be equal to an electrical current peak of about 100 μA for a child. In an exemplary embodiment, the second threshold electrical current value may be equal to an electrical current peak of about 450 μA for an adult person. In an exemplary embodiment, the second threshold electrical current value may be equal to an electrical current peak of about 300 μA for a child.

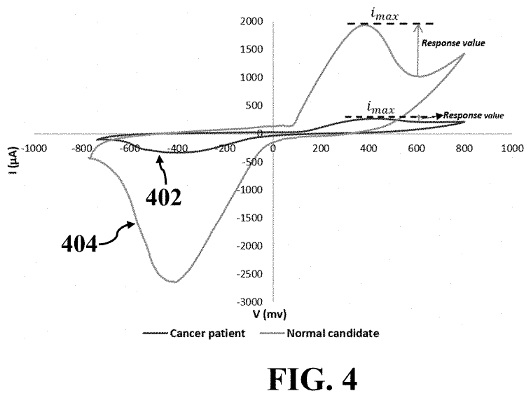

Referring back to FIGS. 2A and 2B, step 212 may include detecting a cancer status of an exemplary suspected person based on the measured ratio of LDNs to HDNs in exemplary unprocessed blood sample 150. FIG. 4 shows comparative cyclic voltammograms of an exemplary cancer patient (diagram 402) and an exemplary healthy candidate (diagram 404), consistent with one or more exemplary embodiments of the present disclosure. In an exemplary embodiment, detecting cancer status of an exemplary suspected person may include detecting a cancer disease in suspected person's body associated with exemplary unprocessed blood sample 150 if the measured ratio of LDNs to HDNs in exemplary unprocessed blood sample 150 is more than a threshold LDNs/HDNs value by detecting the measured maximum electrical current of the measured set of electrical currents being less than the first threshold electrical current value. In an exemplary embodiment, the threshold LDNs/HDNs value may equal to about 1 so that if a level of LDNs is more than HDNs in exemplary unprocessed blood sample 150, a cancer disease may be detected for an exemplary suspected person.

In an exemplary embodiment, the first threshold electrical current value corresponding to a threshold LDNs/HDNs value of 1 may equal to 300 μA for adults and 100 μA for children. In an exemplary embodiment, “adults” as used herein may refer to people who are 18 years old or older. In an exemplary embodiment, “children or pediatrics cohort” as used herein may refer to people who are younger than 18 years, for example, children from new born to 13 years old. In an exemplary embodiment, detecting the cancer disease in the suspected person's body may include detecting the cancer disease in an adult suspected person's body if the measured maximum electrical current of the measured set of electrical currents is less than about 300 μA corresponding to a ratio of LDNs to HDNs in exemplary unprocessed blood sample 150 being more than 1. In another exemplary embodiment, detecting the cancer disease in the suspected person's body may include detecting the cancer disease in a suspected child's body if the measured maximum electrical current of the measured set of electrical currents is less than about 100 μA corresponding to a ratio of LDNs to HDNs in exemplary unprocessed blood sample 150 being more than 1.

In another exemplary embodiment, detecting cancer status of an exemplary suspected person may include detecting no cancer disease (a normal or healthy status) in the suspected person's body if the measured ratio of LDNs to HDNs in exemplary unprocessed blood sample 150 is less than the threshold LDNs/HDNs value. In an exemplary embodiment, no cancer may be detected for an exemplary suspected person if a level of LDNs is less than HDNs in exemplary unprocessed blood sample 150. In an exemplary embodiment, no cancer may be detected for an exemplary suspected person if a level of ratio of LDNs to HDNs in exemplary unprocessed blood sample 150 is less than about 1 if the measured maximum electrical current of the measured set of electrical currents is more than the second threshold electrical current value. In an exemplary embodiment, detecting no cancer disease (a normal or healthy status) in the suspected person's body may include detecting no cancer disease in the suspected person's body if the suspected person is an adult and if the measured maximum electrical current of the measured set of electrical currents is more than 450 μA corresponding to a ratio of LDNs to HDNs in exemplary unprocessed blood sample 150 being less than 1. In another exemplary embodiment, detecting no cancer disease (a normal or healthy status) in the suspected person's body may include detecting no cancer disease in the suspected person's body if the suspected person is a child and if the measured maximum electrical current of the measured set of electrical currents is more than 300 μA corresponding to a ratio of LDNs to HDNs in exemplary unprocessed blood sample 150 being less than 1.

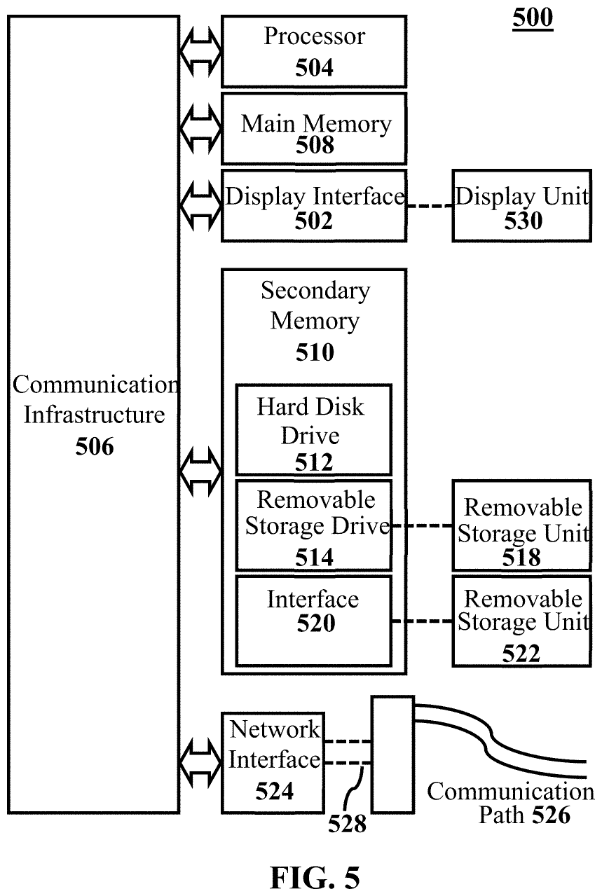

FIG. 5 shows an example computer system 500 in which an embodiment of the present disclosure, or portions thereof, may be implemented as computer-readable code, consistent with one or more exemplary embodiments of the present disclosure. For example, computer system 500 may include an example of processing unit 106 illustrated in FIG. 1, and steps 206-212 of exemplary methods 200 and 220 presented in FIGS. 2A and 2B in addition to steps 232-236 of exemplary method 230 illustrated in FIG. 2C, may be implemented in computer system 500 using hardware, software, firmware, tangible computer readable media having instructions stored thereon, or a combination thereof and may be implemented in one or more computer systems or other processing systems. Hardware, software, or any combination of such may embody any of the modules and components in FIGS. 1, 2A-2C.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the present disclosure is described in terms of this example computer system 500. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multiprocessor machines. In addition, in some embodiments the order of operations may be rearranged without departing from the spirit of the disclosed subject matter.

Processor device 504 may be a special purpose or a general-purpose processor device. As will be appreciated by persons skilled in the relevant art, processor device 504 may also be a single processor in a multi-core/multiprocessor system, such system operating alone, or in a cluster of computing devices operating in a cluster or server farm. Processor device 504 may be connected to a communication infrastructure 506, for example, a bus, message queue, network, or multi-core message-passing scheme.

In an exemplary embodiment, computer system 500 may include a display interface 502, for example a video connector, to transfer data to a display unit 530, for example, a monitor. Computer system 500 may also include a main memory 508, for example, random access memory (RAM), and may also include a secondary memory 510. Secondary memory 510 may include, for example, a hard disk drive 512, and a removable storage drive 514. Removable storage drive 514 may include a floppy disk drive, a magnetic tape drive, an optical disk drive, a flash memory, or the like. Removable storage drive 514 may read from and/or write to a removable storage unit 518 in a well-known manner. Removable storage unit 518 may include a floppy disk, a magnetic tape, an optical disk, etc., which may be read by and written to by removable storage drive 514. As will be appreciated by persons skilled in the relevant art, removable storage unit 518 may include a computer usable storage medium having stored therein computer software and/or data.

In alternative embodiments, secondary memory 510 may include other similar means for allowing computer programs or other instructions to be loaded into computer system 500. Such means may include, for example, a removable storage unit 522 and an interface 520. Examples of such means may include a program cartridge and cartridge interface (such as that found in video game devices), a removable memory chip (such as an EPROM, or PROM) and associated socket, and other removable storage units 522 and interfaces 520 which allow software and data to be transferred from removable storage unit 522 to computer system 500.

Computer system 500 may also include a communications interface 524. Communications interface 524 allows software and data to be transferred between computer system 500 and external devices. Communications interface 524 may include a modem, a network interface (such as an Ethernet card), a communications port, a PCMCIA slot and card, or the like. Software and data transferred via communications interface 524 may be in the form of signals, which may be electronic, electromagnetic, optical, or other signals capable of being received by communications interface 524. These signals may be provided to communications interface 524 via a communications path 526. Communications path 526 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 518, removable storage unit 522, and a hard disk installed in hard disk drive 512. Computer program medium and computer usable medium may also refer to memories, such as main memory 508 and secondary memory 510, which may be memory semiconductors (e.g. DRAMs, etc.).

Computer programs (also called computer control logic) are stored in main memory 508 and/or secondary memory 510. Computer programs may also be received via communications interface 524. Such computer programs, when executed, enable computer system 500 to implement different embodiments of the present disclosure as discussed herein. In particular, the computer programs, when executed, enable processor device 504 to implement the processes of the present disclosure, such as the operations in method 200 illustrated by FIGS. 2A-2C, discussed above. Accordingly, such computer programs represent controllers of computer system 500. Where an exemplary embodiment of method 200 is implemented using software, the software may be stored in a computer program product and loaded into computer system 500 using removable storage drive 514, interface 520, and hard disk drive 512, or communications interface 524.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

### Example 1: Sensor Fabrication Process

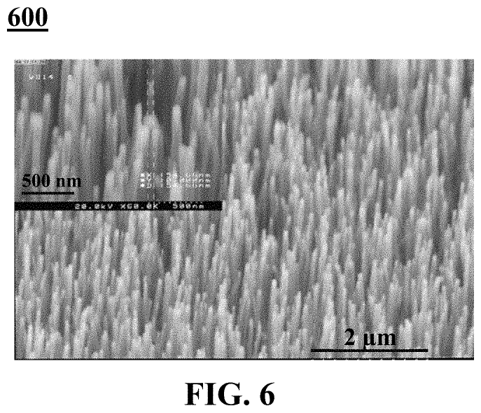

In this example, an exemplary sensor similar to sensor 102 was fabricated via a method similar to exemplary method 300 illustrated in FIGS. 3A and 3B hereinabove. First, p-type Si wafer (100) was cleaned by the standard RCA method (RCA #1 method with NH₄OH:H₂O₂:H₂O solution and a volume ratio of 1:1:5), rinsed in deionized (DI) water, and dried by compressed air. A thermal oxide was then grown on the wafer in a wet oxide furnace at about 1050° C. for about 2.5 hours to form a substrate. Then, a Ni catalyst layer for CNT growth with a thickness of about 9 nm was deposited on the substrate by E-beam evaporation system, at a temperature of about 120° C. and with a depositing rate of about 0.1 Angstroms/s. Afterward, The Ni layer was patterned in a shape of three circles with a diameter of about 2 mm in an exemplary conformation of three electrodes, as working, counter, and reference with a triangular distance of 5 mm from each other using standard photolithography process. For this process, a thin layer of a positive photoresist (e.g., Microchem-S1813) was spin-coated on the substrate surface. After illumination with the Mask-Aligner system, the substrate chemically developed. Then, a Ni layer in an undesired region (a region except of a region of three electrodes) was etched using a Ni-etchant solution (HNO₃:H₃PO₄:CH₃COOH, 3:3:1), and after that, the photoresist was washed using acetone. The entire substrate was then passivated with a 200 nm oxide layer at 0.9 torr, and 20 W (deposition speed <45 nm min⁻¹). The silicon dioxide layer deposited on the electrodes was removed by immersion of the positive PR-patterned substrate in a buffered oxide etchant (BOE) solution for about 7 s to prepare windows for the formed three-metal thin-film electrodes. Finally, the substrate with the formed electrodes thereon was located in a direct current plasma enhanced chemical vapor deposition (DC-PECVD) system to grow vertically aligned multi-walled carbon nanotubes (VAMWCNTs) on electrodes' heads. The growth of VAMWCNTs has a three-step process named annealing, graining, and growth. At first, the substrate with the formed electrodes was annealed at 680° C. in an H₂ environment with a flow rate of 20 standard cubic centimeters per minute (sccm) for 30 minutes. During the graining, the surface was plasma hydrogenated for 5 minutes with the intensity of 5.5 W·cm⁻², which results in the catalyst graining and formation of Ni Nano-sized islands. In growth step, plasma of C₂H₂ and H₂ mixture with flow rates of 4.5 and 20 sccm was introduced to the chamber for about 20 minutes. Grown CNT arrays on electrodes were characterized by Field Emission Scanning Electron Microscope (FE-SEM). FIG. 6 shows an image 600 of a Field Emission Scanning Electron Microscope (FE-SEM) image of exemplary grown CNTs, consistent with one or more exemplary embodiments of the present disclosure. Length and diameter of nanotubes were ranged from about 2.5 μm to about 5 μm and from about 50 nm to about 70 nm, respectively. CNT arrays were used as the work, counter, and reference electrodes. Then, the passivating layer coated on connecting pads of electrodes were removed with BOE and the connecting pads of electrodes were attached to an electrical connector with three pins by conductive paste to form an exemplary final sensor. Afterwards, the electrical connector was connected to a readout system as an example of electrical stimulator-analyzer device 104 by a noiseless cable that handled all three electrodes.

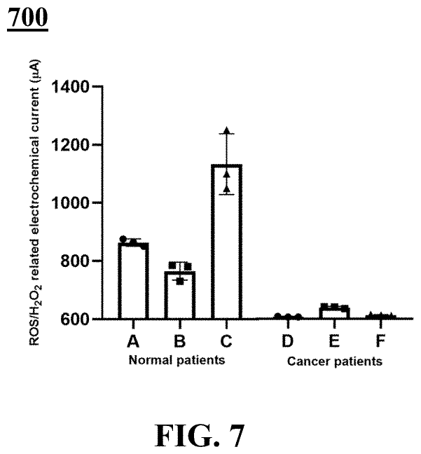

Repeatability of the fabricated sensor was examined by testing three blood samples from normal and cancer patients with a 3-times of test repeat on each of them. FIG. 7 shows a chart 700 representing repeatability of an exemplary fabricated sensor obtained by testing three blood samples from normal and cancer patients with a 3-times of test repeat for each blood sample, consistent with one or more exemplary embodiments of the present disclosure.

### Example 2: Detecting Cancer by Electrochemical Measuring of Reactive Oxygen Species (ROS) Levels in Blood

In this example, one hundred sixty patients of kids (2 months-13 years of age) and adults (20-73 years old) in two groups of newborn cancer with histologically confirmed cancer (n=60) and normal candidates (n=100) were tested here to investigate a correlation between ROS electrochemical peak results and abundancy ratio of HDNs to LDNs in their unprocessed blood samples. All cancer patients had advanced diseases (stages III-IV) in accordance with the American Joint Committee on Cancer criteria. Additionally, some treated patients with chemo/radiotherapy were investigated (n=20). Clinical, laboratory, and radiological characteristics and diagnosis and outcomes data were collected from all patients. Fresh blood samples prepared from 180 candidates were recorded. Also, patients' symptoms and therapeutic steps of cancer patients were recorded. For each patient, about 1 cc blood was used to measure released ROS/H₂O₂ from HDNs and LDNs in peripheral blood utilizing an exemplary system similar to system 100 including an exemplary sensor fabricated in EXAMPLE 1 hereinabove via an exemplary method similar to method 200. Furthermore, LDNs and HDNs were isolated from whole blood with magnetic isolation and Percoll gradient. Then, LDN and HDN cells were counted by cell counter. Also, LDN and HDN cells were stained with Giemsa following thin-layer cell preparation and ROS fluorescent assay of LDNs and HDNs was carried out using a fluorescent microscopy system.

Neutrophil Isolation from Whole Blood and Quantification:

Total circulating neutrophils were isolated from 5 ml cancer patients' blood by negative selection using a Whole Blood Neutrophil Isolation Kit for human. Then, LDN and HDNs were isolated by discontinuous Percoll density gradient method. 9 ml Percoll was added to 1 ml 10×PBS (Stem cell) to create a 100% solution. Then, 2.5 ml of 78% Percoll diluted in 1×PBS was added carefully to a 15 ml falcon tube followed by a 2 ml layer of 66% Percoll and, so total neutrophils resuspended in 2 ml 54% Percoll. Centrifugation at 1545×g without brake was performed for 30 minutes at room temperature. HDNs were recovered from the 78%/66% interface and LDNs from 66%/54% interface. Cell counts were performed using a cell counter. Afterward, HDNs and LDNs cells were fixed by dipping the glass slides in 70% ethanol for 2 min and allowed to dry at room temperature before staining. HDNs and LDNs cells were stained 50 min in Giemsa solution, and washed in Tap water. Finally, glass slides were air-dried shortly and the dried slides were inspected under a light microscope.

Ros Assay:

ROS generation was analyzed with a 5-chloromethyl-2′-7′-dichlorodihydrofluorescein diacetate (CM-H2DCFDA) assay. This probe is changed to 2′-7′-dichlorofluorescein (DCF) with a green fluorescent property by esterase enzymes in the cells' cytosol. After culturing fibroblast and MCF-7 cells overnight, the cells were washed with PBS, and then 500 μL of CM-H2DCFDA solution was added with a concentration of 20 μM. After 30 min incubation at room temperature in dark ambient, cells were again washed and then imaged with a fluorescent microscope. The cells were incubated with 10 mM of NAC as a ROS scavenger and then treated with CM-H2DCFDA. For positive control, cells were incubated with 100 μM H₂O₂ and treated by CM-H2DCFDA. Afterwards, samples were imaged with a fluorescent microscopy system.

An exemplary sensor fabricated in EXAMPLE 1 hereinabove was used to detect ROS levels in about 1 cc of an unprocessed blood sample from cancer patients and normal cases. The unprocessed blood sample was a residue of conventionally received blood from volunteers due to their checkup by informing them and their satisfaction. Intensity of released ROS levels in peripheral blood was recorded by a cyclic voltammetry procedure via a method similar to method 200 described hereinabove. Coherent results were achieved after categorizing the measured ROS current peaks and comparing them with candidates' clinical diagnostic results and abundancy of LDNs versus HDNs in their blood samples. Table 1 shows baseline characteristics of 160 patients investigated herein, including group 1 (G1) of normal candidate/patients with non-cancer diseases and group 2 (G2) of patients with newborns cancer.

TABLE 1
Baseline characteristics of 160 investigated patients (G1: Normal candidate/patients
with non-cancer diseases, G2: Patients with newborns cancer).

| | | | | | State of patients | |
|---|---|---|---|---|---|---|
| | | | | | Normal/Non | |
| | Characteristic | | | | cancer diseases | Malignant tumor |
| Candidates | Age | Sex | | Race | (n = 80) | (n = 80) |
| Children | 2 months-13 | Female | Male | White | Appendicitis | Ewing sarcoma/neck |
| | years old | (n = 21) | (n = 39) | | (n = 5) | tumor mass (n = 3) |
| | | | | | Abdominal | Rhabdomyosarcoma |
| | | | | | edema (n = 6) | pelvis (n = 6) |
| | | | | | Abdominal hernia | Ewing |
| | | | | | (n = 2) | sarcoma/abdomen |
| | | | | | Left kidney stone | tumor mass |
| | | | | | (n = 1) | (n = 4) |
| | | | | | Liver Hydatid | Brain tumor in the |
| | | | | | cyst (n = 3) | left ventricle (n = 2) |
| | | | | | Hypertrophic | Lung cancer (n = 3) |
| | | | | | pyloric stenosis | Wilms tumor (n = 7) |
| | | | | | (n = 2) | Hepatoblastoma |
| | | | | | Osteocyte foot | (n = 2) |
| | | | | | bone (n = 2) | Neuroblastoma |
| | | | | | Vesicostomy | (n = 2) |
| | | | | | (n = 3) | Osteosarcoma (n = 1) |
| | | | | | Normal (n = 6) | |
| Adults | 20-73 | Female | Male | White | Fibrocystic | Phyllodes |
| | | (n = 100) | (n = 0) | | changes/ | (cancerous) (n = 5) |
| | | | | | Microcalcification | IDC nuclear grade 2 |
| | | | | | (n = 20) | (n = 60) |
| | | | | | Fibroadenoma/ | IDC nuclear grade 3 |
| | | | | | Cystic mass | (n = 25) |
| | | | | | (n = 10) | high grade DCIS |
| | | | | | Florid ductal | (n = 7) |
| | | | | | hyperplasia | Low/inter mediate |
| | | | | | (n = 20) | grade DCIS (n = 2) |
| | | | | | Normal (n = 50) | ILC (n = 1) |

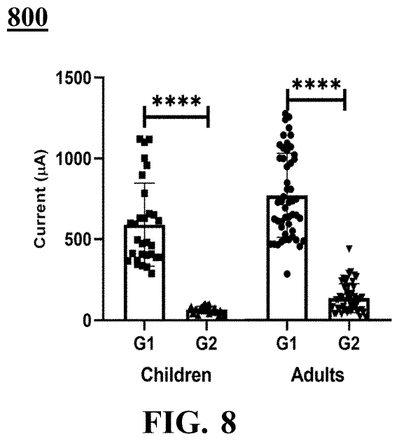

Also, a calibration pattern was provided between responses measured and obtained by exemplary fabricated sensor and results of pathological state of the patients with tumors. FIG. 8 shows a chart 800 of measured peak currents associated with ROS levels in blood samples of 160 investigated patients for two groups of G1: normal candidate/patients with non-cancer diseases and G2: patients with newborns cancer, consistent with one or more exemplary embodiments of the present disclosure. As may be seen in FIG. 8, 100% of blood samples in all children with advanced cancers (stages III-IV) showed peak currents lower than about 100 μA, while normal and non-cancerous candidates showed current peaks higher than about 300 μA. These ROS/H₂O₂ assisted current peaks for 90% of adults intended in this study with breast cancer tumors were lower than about 300 μA. In comparison, 98% of normal candidates with no complaint cases (including healthy people or diagnosed benign tumors) showed peak currents higher than about 450 μA. As a result, the ROS/H₂O₂ assisted electrochemical current peaks of the blood sample in patients with cancer tumors shows significant differences to blood current peaks of the normal/none cancer patients. This gap in pediatrics cohort is about 200 μA so that a ROS current peak for normal cases is more than about 300 μA, whereas a ROS current peak for cancer patients is less than about 100 μA. A gap for adults is about 150 μA so that a ROS current peak for normal cases is more than about 450 μA, whereas a ROS current peak for cancer patients is less than about 300 μA. So, the cut-off between normal/non-cancer disease and cancer cases is well detectable. It should be mentioned that adult cancer cohorts only were selected from breast cancer patients due to available clinical research center's facility.

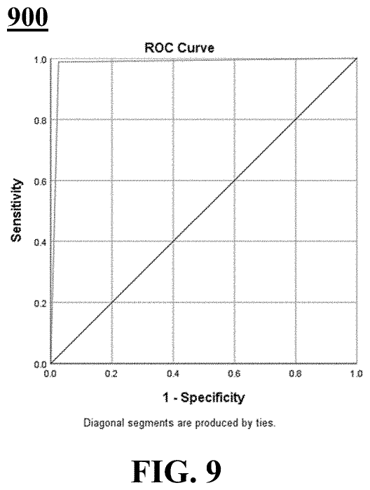

Furthermore, receiver operating characteristic (ROC) curve analysis was done to compare between peak level responses associated with neutrophil ROS production in blood measured by exemplary fabricated sensor and results of clinical diagnostics (gold standard). The clinical diagnostics, including pathological and radiological evaluation, was considered as the gold standard test for diagnosis cancerous specimens. FIG. 9 shows a ROC diagram 900 for results obtained by utilizing exemplary method and fabricated sensor versus clinical diagnostics results for blood samples from 160 patients, consistent with one or more exemplary embodiments of the present disclosure. The result showed that an area under ROC curve (AUC) value for exemplary method 200 or method 220 of the present disclosure utilizing exemplary system 100 and sensor 102 was 0.981 (with P-value of less than about 0.0001 and CI99% 0.949-1) as represented in FIG. 9 and Table 2. Therefore, the obtained AUC value within a range of 0.9 to 1.0 may be an indicator of very high accuracy of exemplary method, system, and fabricated sensor which shows that exemplary method, system, and fabricated sensor is a reliable diagnostic tool and has a good balance of sensitivity and specificity.

Moreover, specificity, sensitivity, accuracy, precision, and selectivity was calculated for exemplary method and fabricated sensor as presented in Table 3. Also, true and false positive and negative data are shown in detail in Table 3. Accordingly, exemplary method, system, and sensor described herein can be used as an accurate diagnostic tool for detecting cancer in suspicious cases by real-time testing of peripheral blood. Exemplary fabricated sensor showed selectivity of about 98% and specificity of about 96% for the presence of cancer in patients with a breast mass and no signs of other inflammatory diseases (which may activate cytokine storm). In conclusion, exemplary fabricated sensor has proper sensitivity and specificity and can be used as a diagnostic tool for detecting suspicious cases by real-time testing of peripheral blood.

TABLE 2
AUC for exemplary fabricated sensor and method versus clinical
diagnostics results for blood samples from 160 patients
Area Under the Curve
Test Result Variable(s) for exemplary fabricated sensor

| | | Asymptotic | Asymptotic 99% Confidence Interval | |
|---|---|---|---|---|
| Area | Std. Error^{a} | Sig.^{b} | Lower Bound | Upper Bound |
| 0.981 | 0.012 | 0.000 | 0.949 | 1.000 |

The test result variable(s): Exemplary fabricated sensor has at least one tie between the positive actual state group and the negative actual state group. Statistics may be biased.
^{a}Under the nonparametric assumption
^{b}Null hypothesis: true area = 0.5

TABLE 3
Cross tabulation results for exemplary fabricated
sensor, method, and system versus clinical diagnostics
as a gold standard for blood samples from 160 patients
Exemplary system/method versus Clinical diagnostics Crosstabulation

| | | | Clinical | | |
|---|---|---|---|---|---|
| | | | diagnostics | | |
| | | | Nega- | Posi- | |
| | | | tive | tive | Total |
| Exemplary | Negative | Count | 77 | 1 | 78 |
| system/ | | % within exemplary | 98.7% | 1.3% | 100.0% |
| method | | system/method | | | |
| | | % within Clinical | 97.5% | 1.2% | 48.8% |
| | | diagnostics | | | |
| | Positive | Count | 2 | 80 | 82 |
| | | % within exemplary | 2.4% | 97.6% | 100.0% |
| | | system/method | | | |
| | | % within Clinical | 2.5% | 98.8% | 51.2% |
| | | diagnostics | | | |
| Total | | Count | 79 | 81 | 160 |
| | | % within exemplary | 49.4% | 50.6% | 100.0% |
| | | system/method | | | |
| | | % within Clinical | 100.0% | 100.0% | 100.0% |
| | | diagnostics | | | |

Sensitivity = 98.80%,
Specificity = 97.50%,
Accuracy = 98.70%,
Precision = 97.60%,
Selectivity = 96.33%

In addition, isolation and characterization of high-density and low-density Neutrophils in comparison with ROS florescent assay was done to check validity of exemplary method described and conducted herein based on electrochemical measurement of ROS levels in blood samples utilizing exemplary fabricated probe hereinabove. Isolation and counting a ratio of LDNs versus HDNs in patients' blood may reveal better a relation between sensor scores and pathological states of blood volunteers. Mature neutrophils are characterized by a polymorphonuclear nucleus, which is in general large with 2-5 lobes ‘segmented neutrophils’, whereas immature neutrophils are shown by a one-lobed curved or ring-shaped nucleus. LDNs (immature and some mature neutrophils) are more in blood of cancer patients.

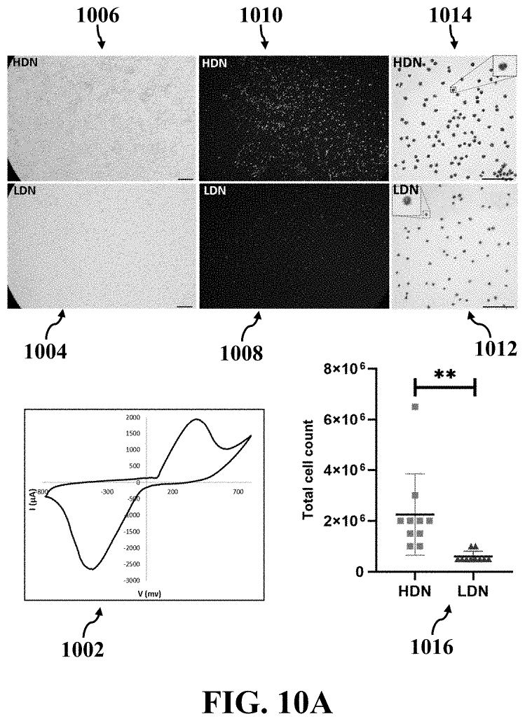

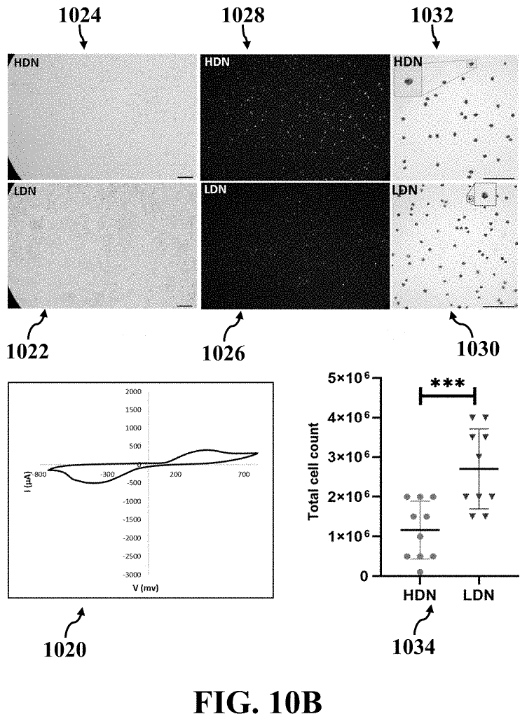

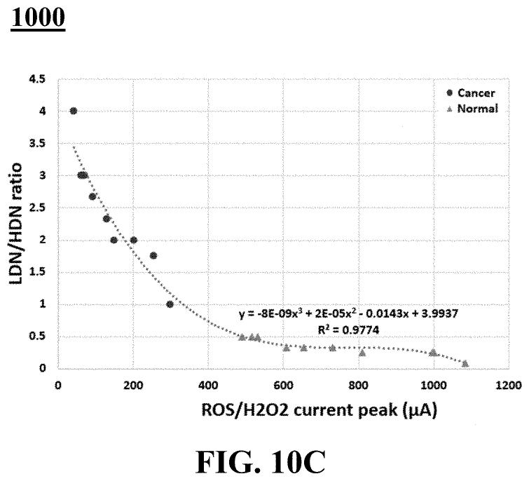

FIG. 10A shows representative images of ROS/H₂O₂ related electrochemical current peak 1002, optical microscope images of LDNs 1004 and HDNs 1006, fluorescent microscope images of LDNs 1008 and HDNs 1010, Giemsa staining images of LDNs 1012 and HDNs 1014, and total cell count 1016 of LDNs and HDNs isolated by magnetic, negative selection protocol neutrophils isolated from 5 ml blood of normal cases (N=10), consistent with one or more exemplary embodiments of the present disclosure. Furthermore, FIG. 10B shows representative images of ROS/H₂O₂ related electrochemical current peak 1020, optical microscope images of LDNs 1022 and HDNs 1024, fluorescent microscope images of LDNs 1026 and HDNs 1028, Giemsa staining images of LDNs 1030 and HDNs 1032, and total cell count 1034 of LDNs and HDNs isolated by magnetic, negative selection protocol neutrophils isolated from 5 ml blood of cancer patients (N=10), consistent with one or more exemplary embodiments of the present disclosure. A P-value less than 0.05 was considered significant. Data shows means±SEM error bars. The bar in optical microscope images and Giemsa staining images represents 100 μm. As shown in FIGS. 10A and 10B, ROS peak levels recorded by exemplary method and system described herein are correlated well with an elevated ratio of LDNs/HDNs in normal and cancer patients as presented in detail in Table 4.

Fluorescent ROS assay may reveal a correlation between ROS/Hypoxic functions of cultured LDNs and HDNs and their recorded current peaks. As shown in FIGS. 10A and 10B, ROS fluorescent intensity showed weaker expression of florescent tagged cells in patients' blood with invasive and malignant cancer versus normal candidates or patients with non-cancer diseases. A direct correlation between down-regulation of ROS fluorescent images and electrochemical measured peaks was observed in all cancer blood samples.

TABLE 4
Detailed information of HDN and LDN counts and measured current peaks in
20 normal cases (N = 10) and cancer patients (N = 10)

| | | HDN cell | LDN cell | | current |
|---|---|---|---|---|---|
| | | counts in | counts in | | peak (μA) |
| Patient | | 5 ml of | 5 ml of | LDN/HDN | for 1 ml of |
| ID | State of patients | blood | blood | ratio | blood |
| 1 | Normal | 2*10⁶ | 0.5*10⁶ | 0.25 | 1000 |
| 2 | Normal | 1.5*10⁶ | 0.5*10⁶ | 0.33 | 730 |
| 3 | Normal | 2*10⁶ | 0.5*10⁶ | 0.25 | 810 |
| 4 | Normal | 2*10⁶ | 1*10⁶ | 0.5 | 531 |
| 5 | Cancer (IDC nuclear grade 2) | 2*10⁶ | 3.5*10⁶ | 1.75 | 255 |
| 6 | Cancer (IDC nuclear grade 2) | 1.5*10⁶ | 4*10⁶ | 2.67 | 92 |
| 7 | Cancer (IDC nuclear grade 2) | 1*10⁶ | 2*10⁶ | 2 | 149 |
| 8 | Cancer (IDC nuclear grade 3) | 0.5*10⁶ | 1.5*10⁶ | 3 | 61 |
| 9 | Cancer (IDC nuclear grade 3) | 0.5*10⁶ | 1.5*10⁶ | 3 | 71 |
| 10 | Normal | 1.5*10⁶ | 0.5*10⁶ | 0.33 | 609 |
| 11 | Normal | 1*10⁶ | 0.5*10⁶ | 0.5 | 489 |
| 12 | Normal | 6.5*10⁶ | 0.5*10⁶ | 0.08 | 1085 |
| 13 | Cancer (IDC nuclear grade 3) | 2*10⁶ | 2*10⁶ | 1 | 298 |
| 14 | Cancer (IDC nuclear grade 2) | 1.5*10⁶ | 3.5*10⁶ | 2.33 | 129 |
| 15 | Cancer (IDC nuclear grade 2) | 2*10⁶ | 4*10⁶ | 2 | 202 |
| 16 | Normal | 2*10⁶ | 0.5*10⁶ | 0.25 | 997 |
| 17 | Cancer (IDC nuclear grade 3) | 1*10⁶ | 3*10⁶ | 3 | 65 |
| 18 | Normal | 3*10⁶ | 1*10⁶ | 0.33 | 654 |
| 19 | Normal | 1*10⁶ | 0.5*10⁶ | 0.5 | 515 |
| 20 | Cancer (IDC nuclear grade 3) | 0.5*10⁶ | 2*10⁶ | 4 | 41 |

FIG. 10C shows a chart 1000 of LDN/HDN ratio versus measured current peak diagram with a fitted plot for 20 cases, including normal cases (N=10) and cancer patients (N=10), consistent with one or more exemplary embodiments of the present disclosure. It may be observed that a level of LDN/HDN ratio may be precisely determined by real-time measuring electrochemical current peak of a 1 cc blood sample drawn from a person utilizing exemplary method, system, and fabricated sensor described herein.

Percentage Changes of ROS Level and LDNs/HDNs Ratio:

Herein, percentage of changes of ROS level and LDNs/HDNs ratio for cases investigated hereinabove (reported in Table 4) was calculated according to Equations (1) and (2) herein below.

$$ROS level changes in cancer patient ’ blood ( \% ) = \frac{\begin{matrix}- ( average blood current peaks of cancer patients - \\ average blood current peaks of Normal cadidiates )\end{matrix}}{average blood current peaks of Normal candidates} \times 100 \qquad Equation ( 1 )$$

ROS level changes in cancer patient' blood (%) according to data summarized in Table 4 using Equation (1) is calculated as follow:

$$ROS level changes in cancer patient ’ blood ( \% ) = \frac{- ( 136.3 - 742 )}{742} \times 100 = 82 \% \qquad Equation ( 2 )$$

$$LDNs / HDNs ratio changes in cancer patients ’ blood ( \% ) = \frac{\begin{matrix}- ( average \frac{LDNs}{HDNs} ratio of cancer patients - \\ average \frac{LDNs}{HDNs} ratio of Normal cadidates )\end{matrix}}{average LDNs / HDNs ratio of Normal candidates} \times 100$$

LDNs/HDNs ratio changes in cancer patients' blood (%) according to data summarized in Table 4 using Equation (2) is calculated as follow:

$$LDNs / HDNs ratio chanes in cancer patients ’ blood ( \% ) = \frac{( 2.475 - 0.332 )}{0.332} \times 1 \; 00 = 65 \%$$

A significantly reduced ROS (82%) level in peripheral blood of cancer patients measured by an exemplary fabricated electrochemical sensor, which showed a great correlation with an increased ratio of LDNs/HDNs (65%) in such patients was observed.

### Example 3: Effect of Chemo/Radiotherapy on Patients' Blood and Sensor Response

Table 5 shows detailed information of 20 patients with chemo/radiotherapy and measured electrochemical current peaks utilizing exemplary system, method, and fabricated sensor described hereinabove. As may be observed in Table 5, blood samples of non-treated patients with invasive tumors showed significantly lower levels of CV peaks than their blood after complete response (due to an associated oncologist's opinion) to chemo/radiotherapy. It revealed that exemplary system, method, and fabricated sensor has a capability of monitoring therapeutic effects on cancer patients by testing a 1 cc blood sample drawn from a patient. As COVID-19 may also increase Neutrophils' levels in blood due to cytokine storm, which may perturb cancer-associated response of exemplary sensor and method here, RT-PCR test and clinical evaluation was done on patients to be ensured from non-COVID involvement in them.

TABLE 5
Detailed information of 20 patients with chemo/radiotherapy
and associated measured electrochemical current peaks.

| | | Therapy | current peak |
|---|---|---|---|
| Patient | | (Surgery, Chemo/ | (μA) for 1 ml |
| ID | Cancer type | radiotherapy (session)) | of blood |
| 161 | IDC nuclear grade 3 | Chemo (8) and Surgery (mastectomy) | 1090 |
| 162 | IDC nuclear grade 2 | Chemo (5) | 550 |
| 163 | IDC nuclear grade 2/3 | Chemo (6)/Radio (30) and surgery | 939 |
| | | (lumpectomy) | |
| 164 | IDC nuclear grade 3 | Chemo (6)/Radio (28) | 731 |
| 165 | IDC nuclear grade | Chemo (8) | 469 |
| | 2/DCIS component | | |
| | (20%) | | |
| 166 | IDC nuclear grade 3 | Chemo (6)/Radio (0) | 393 |
| 167 | IDC nuclear grade 2/3 | Chemo (4)/Radio (0) | 321 |
| 168 | IDC nuclear grade 2 | Chemo (5) and Surgery (mastectomy) | 498 |
| 169 | IDC nuclear grade 2 | Chemo (8) | 681 |
| 170 | IDC nuclear grade 2/3 | Chemo (6)/Radio (20) | 537 |
| 171 | IDC nuclear grade 3 | Chemo (6) and Surgery (mastectomy) | 786 |
| 172 | IDC nuclear grade | Chemo (6) | 606 |
| | 2/DCIS | | |
| 173 | IDC nuclear grade 3 | Chemo (8) | 815 |
| 174 | IDC nuclear grade 2 | Surgery (lumpectomy) and radio (0) | 495 |
| 175 | IDC nuclear grade 2 | Surgery (lumpectomy) | 562 |
| 176 | IDC nuclear grade 2 | Surgery (lumpectomy) | 510 |
| 177 | IDC nuclear grade 3 | Chemo (8) | 840 |
| 178 | IDC nuclear grade 3 | Chemo (4) | 325 |
| 179 | IDC nuclear grade 2 | Chemo (6) and Surgery (mastectomy) | 595 |
| 180 | IDC nuclear grade 2/3 | Chemo (8) and Surgery (mastectomy) | 700 |

### Example 4: Effect of Other Diseases on Sensor Response

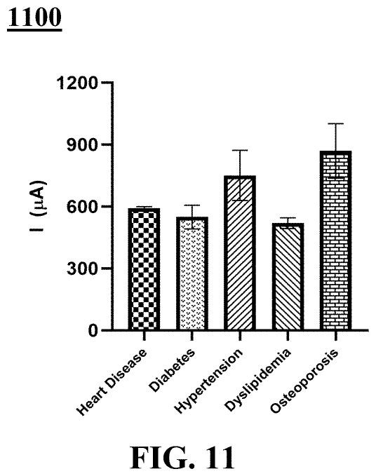

Different kinds of pediatrics sickness with non-cancer diseases and normal candidates were studied as presented in Table 1. For adult investigation, electrochemical ROS detection was also performed on non-breast cancer diseases. Moreover, blood ROS levels were recorded in some patients with hypertension, heart disease, dyslipidemia, osteoporosis, and diabetes who had no tumoral diseases. FIG. 11 shows a chart 1100 of ROS/H₂O₂ assisted electrochemical current peaks of blood samples of patients with non-cancer diseases (n=15), consistent with one or more exemplary embodiments of the present disclosure. As shown in FIG. 11, results showed peak currents higher than about 450 μA in all cases. This means that the ROS/H₂O₂ assisted electrochemical current peaks of the blood sample in patients with non-cancer diseases are significantly different from blood current peaks of the cancer patients. ROS/H₂O₂ assisted electrochemical current peaks in normal and non-cancer diseases are more than about 450 μA while in cancer diseases are less than about 300 μA.

LDNs may be increased in blood due to cancer, infection, and inflammation. One of the inflammatory diseases is COVID-19 as a dangerous viral pandemic. It is often more severe in people over 60 years old or with health conditions like lung or heart disease, diabetes, or conditions that affect their immune system. In COVID-19 patients, a significant increase of immature neutrophil numbers may be observed in acute patients compared to healthy donors/recovered patients, while the number of mature neutrophils decreased. Also, a meaningful shift in a ratio between mature and immature neutrophils may associate with a severity of viral involvement. The presence of “low density inflammatory neutrophils” may be strongly correlated with disease severity, and IL-6 levels. In addition, immature neutrophil numbers may strongly associate with IL-6 and IP-10. IL-6 and IP-10 may be consistently upregulated during cytokine storms and may be correlated with severe acute respiratory distress syndrome (ARDS). In addition to inflammatory monocytes as a source of IL-6, immature neutrophils may also be a non-negligible source of IL-6 through COVID-19 induced cytokine storm. So, an increase in number of LDNs may occur in cytokine storm stage of COVID disease. To evaluate an effect of COVID-19 as the most frequent infectious disease currently on peripheral blood ROS, exemplary system, method, and fabricated sensor was tested on blood sample of 14 patients with COVID-19 who were hospitalized in the intensive care unit (ICU).

Table 6 shows electrochemical ROS peak currents measured for blood samples of COVID-19 infected patients with any comorbidity. ROS/H₂O₂ assisted current peaks for 87% of adults with COVID-19 infection were lower than about 300 μA, same as patients with breast cancer tumors. But it is worth noting that when a patient comes to a medical center for evaluation of her/his breast tumor, COVID-19 involvement is prechecked for her/him, and global vaccination drastically reduces a probability of COVID-19 involvement with cytokine storm in the people. Hence, herein candidates suspicious to have high-risk breast tumors with no trace of inflammatory diseases or pregnancy were evaluated.

TABLE 6
Electrochemical ROS peak currents in COVID-19
infected patients with any comorbidity.

| Patient | | | Past disease | | | | RT- | CT- | I |
|---|---|---|---|---|---|---|---|---|---|
| ID | Age | Gender | history | Symptoms | ESR | CRP | PCR | Scan | (μA) |
| 1 | 61 | Male | Lung | Dyspnoea, | 42 | 24.7 | Pos. | Pos. | 125 |
| | | | cancer/DM | Unconscious | | | | | |
| 2 | 72 | Male | Colon | Myalgia or | 16 | 20.3 | Pos. | Pos. | 228 |
| | | | cancer | fatigue, | | | | | |
| | | | | Dyspnoea, | | | | | |
| | | | | Lack of | | | | | |
| | | | | appetite | | | | | |
| 3 | 63 | Male | IHD, CVA, | loss of | 29 | 31.3 | Pos. | Pos. | 355 |
| | | | DVT | consciousness, | | | | | |
| | | | | hypoxemia | | | | | |
| 4 | 79 | Male | IHD, DM, | Fever, | 24 | 29.5 | Pos. | Pos. | 149 |
| | | | Alzheimer's | Myalgia or | | | | | |
| | | | | fatigue, loss of | | | | | |
| | | | | consciousness | | | | | |
| 5 | 73 | Female | PI, HLP, | Fever, | 27 | 21.3 | Pos. | Pos. | 194 |
| | | | DM, HTN, | Myalgia or | | | | | |
| | | | DLP | fatigue, loss of | | | | | |
| | | | | consciousness | | | | | |
| 6 | 48 | Male | HLP, | Fever, | 34 | 28.6 | Pos. | Pos. | 202 |
| | | | PMH, IHD, | Myalgia or | | | | | |
| | | | DM | fatigue, | | | | | |
| | | | | Dyspnoea, | | | | | |
| | | | | Chest pain | | | | | |
| 7 | 67 | Male | DM, | Fever, | 35 | 20.5 | Pos. | Pos. | 125 |
| | | | Parkinson | Myalgia or | | | | | |
| | | | | fatigue, | | | | | |
| | | | | Vomit, loss of | | | | | |
| | | | | consciousness | | | | | |
| 8 | 73 | Male | HTN, | Fever, Cough, | 10 | 14 | Pos. | Pos. | 157 |
| | | | Pneumonia | Dyspnoea, | | | | | |
| | | | | Diarrhoea | | | | | |
| 9 | 58 | Female | DM, HTN | Fever, Cough, | 20 | 25.8 | Pos. | Pos. | 348 |
| | | | | loss of | | | | | |
| | | | | consciousness | | | | | |
| 10 | 67 | Male | DM, HTN, | Fever, Cough, | 26 | 22.5 | Pos. | Pos. | 125 |
| | | | | Chest pain | | | | | |
| 11 | 62 | Male | DM | Fever, | 34 | 26.5 | Pos. | Pos. | 149 |
| | | | | Dyspnoea, | | | | | |
| | | | | fatigue, loss of | | | | | |
| | | | | consciousness | | | | | |
| 12 | 59 | Female | IHD, HTN | Fever, fatigue, | 17 | 20 | Pos. | Pos. | 251 |
| | | | | chest pain | | | | | |
| 13 | 70 | Male | DM, | Fever, | 15 | 19.3 | Pos. | Pos. | 205 |
| | | | Parkinson | Dyspnoea, | | | | | |
| | | | | Chest pain | | | | | |
| 14 | 73 | Female | IHD, DM, | Fever, | 32 | 27.3 | Pos. | Pos. | 188 |
| | | | Alzheimer's | Dyspnoea, | | | | | |
| | | | | loss of | | | | | |
| | | | | consciousness | | | | | |

IHD: ischemic heart disease, CVA: Cerebrovascular accident, DVT: Deep vein thrombosis, PI: primary immunodeficiency, HLP: Hyperkeratosis lenticularis perstans, DLP: Dyslipidemia, PMH: Progressive macular hypomelanosis, DM: Diabetes mellitus, and HTN: Hypertension.

Furthermore, ESR and CRP (blood tests for detecting inflammation) were investigated in breast cancer patients. ROS peak levels recorded by exemplary system described here, ESR and CRP of these patients are shown in Table 7.

TABLE 7
Electrochemical ROS peak currents in
patients with breast cancer tumor

| Patient | | | | ESR* | CRP** | I |
|---|---|---|---|---|---|---|
| ID | Age | Gender | Tumor type | (mm/hr.) | (mg/L) | (μA) |
| 1 | 39 | Female | ILC | 22 | 7 | 118 |
| 2 | 38 | Female | Papillary with | 40 | 1.9 | 106 |
| | | | Atypia | | | |
| 3 | 38 | Female | IDC | 10 | 2.2 | 134 |
| 4 | 38 | Female | DCIS | 20 | 3 | 102 |
| 5 | 54 | Female | IDC | 18 | 4 | 96 |
| 6 | 40 | Female | IDC | 34 | 10 | 89 |
| 7 | 55 | Female | IDC | 7.3 | 2 | 97 |
| 8 | 46 | Female | IDC | 0.5 | 1 | 104 |
| 9 | 41 | Female | IDC | 5.4 | 5 | 124 |
| 10 | 43 | Female | IDC | 11 | 3 | 128 |
| 11 | 39 | Female | IDC | 14 | 0.5 | 171 |
| 12 | 72 | Female | IDC | 19 | 2 | 202 |
| 13 | 39 | Female | IDC | 21 | 3 | 123 |
| 14 | 50 | Female | IDC | 41 | 6 | 178 |
| 15 | 42 | Female | IDC | 45 | 8 | 147 |

*The normal range of ESR is 0-29 mm/hr for women
**The normal range of CRP is less than 10 mg/L

Hence, patients with observable mass in their breast but asymptomatic signature about other inflammatory diseases would-be a candidate for exemplary system and method described here to detect their LDNs associated ROS levels in blood in favor of their tumor disease stage. In addition, a cytokine storm occurs when a patient has symptoms. However, there is a tumor in cancer and the suspected person who is referred for diagnosis has no symptoms in favor of inflammatory disease.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments. This is for purposes of streamlining the disclosure, and is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separately claimed subject matter.

While various embodiments have been described, the description is intended to be exemplary, rather than limiting and it will be apparent to those of ordinary skill in the art that many more embodiments and embodiments are possible that are within the scope of the embodiments. Although many possible combinations of features are shown in the accompanying figures and discussed in this detailed description, many other combinations of the disclosed features are possible. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached claims and their equivalents. Also, various modifications and changes may be made within the scope of the attached claims.

What is claimed is:

1. A system for real-time detecting cancer by analyzing unprocessed blood, comprising:
a sensor, comprising:
a substrate; and
three electrodes formed on the substrate, the three electrodes comprising a working electrode, a counter electrode, and a reference electrode, each respective electrode comprising:
an electrically conductive layer deposited on the substrate, the electrically conductive layer comprising a proximal end and a circular distal end; and
an array of multi-walled carbon nanotubes (VAMWCNTs) grown on the circular distal end, the array of VAMWCNTs configured to be put in contact with an unprocessed blood sample dropped on surface of the sensor, the unprocessed blood sample being drawn from a suspected person to have cancer;
an electrical stimulator-analyzer device electrically connected to the sensor at the respective proximal end of each respective electrode of the three electrodes, the stimulator-analyzer device configured to:
apply a set of voltages in a sweeping range from −0.8 V to +0.8 V to the sensor comprising the VAMWCNTs being in contact with the unprocessed blood sample; and
measure a produced set of electrical currents of the sensor responsive to the applied set of voltages; and
a processing unit electrically connected to the electrical stimulator-analyzer device, the processing unit comprising:
a memory having processor-readable instructions stored therein; and
a processor configured to access the memory and execute the processor-readable instructions, which, when executed by the processor configures the processor to perform a method, the method comprising:
applying, utilizing the stimulator-analyzer device, the set of voltages in the sweeping range from −0.8 V to +0.8 V between the reference electrode and the working electrode;
measuring, utilizing the stimulator-analyzer device, the produced set of electrical currents between the counter electrode and the working electrode;
measuring a level of a ratio of low-density neutrophils (LDNs) to high-density neutrophils (HDNs) in the unprocessed blood sample by measuring a level of reactive oxygen species (ROS) in the unprocessed blood sample, measuring the level of ROS in the unprocessed blood sample comprising measuring a maximum electrical current of the measured set of electrical currents; and
detecting cancer status of the suspected person, comprising:
detecting a cancer disease in suspected person's body if the measured ratio of LDNs to HDNs in the unprocessed blood sample is more than 1 by detecting the measured maximum electrical current of the measured set of electrical currents being less than a first threshold electrical current value; or
detecting no cancer disease in the suspected person's body if the measured ratio of LDNs to HDNs in the unprocessed blood sample is less than 1 by detecting the measured maximum electrical current of the measured set of electrical currents being more than a second threshold electrical current value.

2. The system of claim 1, wherein detecting the cancer disease in the suspected person's body comprises one of:
detecting the cancer disease in the suspected person's body if the suspected person's body is an adult responsive to the measured maximum electrical current of the measured set of electrical currents being less than 300 μA corresponding to a ratio of LDNs to HDNs in the unprocessed blood sample being more than 1; or
detecting the cancer disease in the suspected person's body if the suspected person's body is a child responsive to the measured maximum electrical current of the measured set of electrical currents being less than 100 μA corresponding to a ratio of LDNs to HDNs in the unprocessed blood sample being more than 1.

3. The system of claim 1, wherein detecting no cancer disease in the suspected person's body comprises one of:
detecting no cancer disease in the suspected person's body if the suspected person's body is an adult responsive to the measured maximum electrical current of the measured set of electrical currents being more than 450 μA corresponding to a ratio of LDNs to HDNs in the unprocessed blood sample being less than 1; or
detecting no cancer disease in the suspected person's body if the suspected person's body is a child responsive to the measured maximum electrical current of the measured set of electrical currents being more than 300 μA corresponding to a ratio of LDNs to HDNs in the unprocessed blood sample being less than 1.

4. The system of claim 1, wherein the method further comprises determining the first threshold electrical current value and the second threshold electrical current value, comprising:
generating a first dataset of a plurality of unprocessed blood samples associated with a plurality of cancer patients, comprising:
measuring a first set of electrical current peaks of the unprocessed blood samples associated with the plurality of cancer patients;
measuring a first set of ratio of LDNs to HDNs in the unprocessed blood samples associated with the plurality of cancer patients utilizing a cell counter; and
assigning each measured ratio of LDNs to HDNs of the first set of ratio of LDNs to HDNs to the respective measured electrical current peak of the first set of electrical current peaks;
generating a second dataset of a plurality of unprocessed blood samples associated with a plurality of healthy persons, comprising:
measuring a second set of electrical current peaks of the unprocessed blood samples associated with the plurality of healthy persons;
measuring a second set of ratio of LDNs to HDNs in the unprocessed blood samples associated with the plurality of healthy persons utilizing a cell counter; and
assigning each measured ratio of LDNs to HDNs of the second set of ratio of LDNs to HDNs to the respective measured electrical current peak of the second set of electrical current peaks; and
determining the first threshold electrical current value and the second threshold electrical current value, comprising:
determining the first threshold electrical current value equal to a maximum electrical current peak among the first set of electrical current peaks; and
determining the second threshold electrical current value equal to a minimum electrical current peak among the second set of electrical current peaks.

5. The system of claim 1, wherein the method is conducted in less than 30 seconds.

6. The system of claim 1, wherein the circular distal end has a diameter in a range of 0.5 mm to 3 mm.

7. The system of claim 1, wherein the three respective circular distal ends of the three electrodes are placed apart from each other by a distance between 1 mm and 5 mm.

8. The system of claim 1, wherein the substrate comprises a first layer of silicon dioxide deposited on a layer of silicon.

9. The system of claim 8, wherein the sensor further comprises a second layer of silicon dioxide deposited on surface of sensor except surface of the circular distal end and the proximal end of each respective electrode of the three electrodes.

10. The system of claim 1, wherein the electrically conductive layer comprises a layer of at least one of nickel, gold, and combinations thereof.

11. The system of claim 1, wherein the electrically conductive layer has a thickness in a range of 5 nm to 20 nm.

12. The system of claim 1, wherein:
the array of VAMWCNTs comprises VAMWCNTs with a length in a range of 2.5 μm to 5 μm; and
the array of VAMWCNTs comprises VAMWCNTs with a diameter in a range of 50 nm to 70 nm.

13. A method for real-time detecting cancer, comprising:
putting an unprocessed blood sample in contact with sensing parts of a set of a working electrode, a counter electrode, and a reference electrode of a sensor, the unprocessed blood sample drawn from a suspected person to have cancer;
applying a set of voltages in a sweeping range from −0.8 V to +0.8 V between the reference electrode and the working electrode;
measuring, utilizing one or more processors, a produced set of electrical currents between the counter electrode and the working electrode versus the applied set of voltages;
measuring, utilizing one or more processors, a level of ratio of low-density neutrophils (LDNs) to high-density neutrophils (HDNs) in the unprocessed blood sample, comprising:
measuring a level of reactive oxygen species (ROS) in the unprocessed blood sample by measuring a maximum electrical current of the measured set of electrical currents; and
determining the level of ratio of LDNs to HDNs in the unprocessed blood sample, comprising:
detecting the level of ratio of LDNs to HDNs in the unprocessed blood sample is more than 1 if the measured maximum electrical current of the measured set of electrical currents is less than a first threshold electrical current value; and
detecting the level of ratio of LDNs to HDNs in the unprocessed blood sample is less than 1 if the measured maximum electrical current of the measured set of electrical currents is more than a second threshold electrical current value; and
detecting, utilizing one or more processors, cancer status of the suspected person, comprising one of:
detecting a cancer disease in the suspected person's body if the level of ratio of LDNs to HDNs in the unprocessed blood sample is more than one; or
detecting no cancer disease in the suspected person's body if the level of ratio of LDNs to HDNs in the unprocessed blood sample is less than one.

14. The method of claim 13, further comprising generating the first threshold electrical current value and the second threshold electrical current value, comprising:
generating a first dataset of a plurality of unprocessed blood samples associated with a plurality of cancer patients, comprising:
measuring a first set of electrical current peaks of the unprocessed blood samples associated with the plurality of cancer patients;
measuring a first set of ratio of LDNs to HDNs in the unprocessed blood samples associated with the plurality of cancer patients utilizing a cell counter; and
assigning each measured ratio of LDNs to HDNs of the first set of ratio of LDNs to HDNs to the respective measured electrical current peak of the first set of electrical current peaks;
generating a second dataset of a plurality of unprocessed blood samples associated with a plurality of healthy persons, comprising:
measuring a second set of electrical current peaks of the unprocessed blood samples associated with the plurality of healthy persons;
measuring a second set of ratio of LDNs to HDNs in the unprocessed blood samples associated with the plurality of healthy persons utilizing a cell counter; and
assigning each measured ratio of LDNs to HDNs of the second set of ratio of LDNs to HDNs to the respective measured electrical current peak of the second set of electrical current peaks; and
determining the first threshold electrical current value and the second threshold electrical current value, comprising:
determining the first threshold electrical current value equal to a maximum electrical current peak among the first set of electrical current peaks; and
determining the second threshold electrical current value equal to a minimum electrical current peak among the second set of electrical current peaks.

15. The method of claim 13, wherein:
the first threshold electrical current value is 300 μA for an adult person;
the first threshold electrical current value is 100 μA for a child;
the second threshold electrical current value is 450 μA for an adult person; and
the second threshold electrical current value is 300 μA for a child.

16. The method of claim 13, wherein the method is conducted in less than 30 seconds.

17. The method of claim 13, further comprising acquiring the unprocessed blood sample from the suspected person to have cancer.

18. The method of claim 13, wherein putting the unprocessed blood sample in contact with the sensing parts of the set of the working electrode, the counter electrode, and the reference electrode of the sensor comprises putting the unprocessed blood sample in contact with three respective arrays of multi-walled carbon nanotubes (VAMWCNTs) grown on the sensing parts of the set of the working electrode, the counter electrode, and the reference electrode by dropping the unprocessed blood sample on surface of the sensor.

19. The method of claim 13, wherein applying the set of voltages in the sweeping range from −0.8 V to +0.8 V between the reference electrode and the working electrode comprises:
connecting respective proximal ends of the set of the working electrode, the counter electrode, and the reference electrode of the sensor to an electrical stimulator-analyzer device; and
applying the set of voltages in a sweeping range from −0.8 V to +0.8 V to the sensor using the electrical stimulator-analyzer device.

20. The method of claim 19, wherein measuring the produced set of electrical currents between the counter electrode and the working electrode versus the applied set of voltages is done utilizing the electrical stimulator-analyzer device.
